(19) United States
(12) Patent Application Publication
(10) Pub. No.: US 2023/0366891 A1
(43) Pub. Date: Nov. 16, 2023

(54) METHODS AND COMPOSITIONS FOR IN SITU IMMUNE PROFILING OF HEART TRANSPLANT BIOPSIES

(71) Applicant: THE TRUSTEES OF THE UNIVERSITY OF PENNSYLVANIA, Philadelphia, PA (US)

(72) Inventors: Eliot Peyster, Philadelphia, PA (US); Kenneth B. Margulies, Villanova, PA (US); Michael Feldman, Wilmington, DE (US)

(73) Assignee: THE TRUSTEES OF THE UNIVERSITY OF PENNSYLVANIA, Philadelphia, PA (US)

(21) Appl. No.: 18/246,604

(22) PCT Filed: Sep. 24, 2021
(86) PCT No.: PCT/US2021/051911

§ 371 Date: Mar. 24, 2023

Related U.S. Application Data
(60) Provisional application No. 63/083,848, filed on Sep. 25, 2020.

(51) Int. Cl. G01N 33/68

(52) U.S. Cl. CPC G01N 33/6857; G01N 2333/7051; G01N 2333/70517; G01N 2333/7155; G01N 2333/70514; G01N 2333/70532; G01N 2333/70596

(57) ABSTRACT

Methods for predicting risk of heart transplant rejection are disclosed.

### CROSS-REFERENCE TO RELATED APPLICATION

This application claims priority to U.S. Provisional Application No. 63/083,848 filed on Sep. 25, 2020, the entire disclosure being incorporated herein by reference as though set forth in full.

### STATEMENT OF GRANT SUPPORT

This invention was made with government support under TR001880 awarded by the National Institutes of Health. The government has certain rights in the invention.

### FIELD OF THE INVENTION

This invention relates to the fields of immune profiling and heart transplantation rejection. More specifically, the invention provides improved methods for identifying those subjects at increased risk of rejection and/or propensity for heart transplant complications.

### BACKGROUND OF THE INVENTION

Several publications and patent documents are cited throughout the specification in order to describe the state of the art to which this invention pertains. Each of these citations is incorporated herein by reference as though set forth in full.

Heart transplantation has been the treatment of choice for end-stage cardiomyopathy for more than 30 years, with more than 4000 heart transplants performed worldwide each year. Cardiac allograft rejection (CAR) is a serious concern in heart transplant medicine and requires vigilance to identify and treat¹. Post-transplant care is complex, requiring careful titration of vital but potentially dangerous immunosuppressive drugs to mitigate the risk of CAR. The importance of balancing the risk posed by rejection with the risk of the methods used to prevent it is exemplified by the fact that CAR is the leading cause of death in the first year post-transplant, with infectious complications related to immunosuppression representing the second leading cause of death. Because CAR is quite common, occurring in 20-40% of transplant patients in the first year and conferring a significant short and long term risk to allograft survival, frequent surveillance endomyocardial biopsy (EMB) with histologic assessment for rejection has been recommended by heart transplantation guidelines since 1990^{2, 3}.

Poor consistency and accuracy of conventional EMB tissue histologic assessment increases patient risk and is a barrier to achieving precision diagnosis. The guideline-directed histologic analysis of EMB samples for CAR using light microscopy with H&E staining, relies on a qualitative examination for the presence of inflammatory cell infiltrates, estimates of infiltrate extent (focal vs. diffuse), and for the appearance of myocyte damage. Unfortunately, standard histologic grading suffers from poor reliability, with a kappa statistic of 0.39 and a dismal inter-pathologist agreement of 28.4% agreement at the higher grades of rejection (2R and higher) which typically result in major alterations of immunosuppression⁸. Beyond these issues of reliability, the current approach also suffers from a lack of diagnostic accuracy, as defined by the ability of histologic grades to correlate with the clinical trajectories of rejection events. As evidence of the poor specificity of standard histologic analysis, Klingenberg et al withheld treatment in a case series of 17 grade 3A rejections, all of whom experienced benign clinical courses with resolution of histologic rejection on subsequent biopsies¹². Similar findings from the multicenter IMAGE trial also highlight the poor sensitivity of the current standard^{13, 14}. Conventional histologic grading fares no better with regards to sensitivity, as shown in a study by Dandel et al in which nearly half (49%) of n=59 serious clinical rejection events occurred in patients with histologic grade 1R or lower¹⁵.

Post-heart transplant care is highly algorithmic, lacking precision and exposing patients to potential harm. At most centers, it is standard for a heart transplant recipient to undergo 12 or more scheduled surveillance EMBs and at least as many scheduled changes in immunosuppressive regimens in their first year post-transplant alone⁵. Fundamentally, the frequency of the EMB procedure, and indeed the existence of a heavily protocolized medical approach to CAR management in general, results from an inability of providers to employ a reliable, tailored diagnostic and therapeutic approach for each patient based on individual CAR risk. As a result, low CAR risk patients undergo more EMB procedures and more aggressive immunosuppression than needed, while high CAR risk patients undergo less surveillance EMBs and premature weaning of immunosuppression. Both of these scenarios represent opportunities for patient harm through procedural complications, infections, and rejection events, and demonstrate the clear, unmet need for better precision medicine tools for heart transplantation protocols.

### SUMMARY OF THE INVENTION

In accordance with the present invention, a method for detecting an increased propensity for heart transplant rejection, graft dysfunction, or organ failure is disclosed. An exemplary method comprises providing a sample from a subject who has received a transplant from a donor; obtaining an immunophenotype to establish an immune cell infiltration and immune effector profile correlated with risk of serious allograft injury and diagnosing, predicting, or monitoring a transplant status or outcome of the subject who has received the transplant by determining the proportional expression levels of immune cell and effector markers present in said transplant, said markers being at least three of CD3, CD8, FoxP3, IL17, PDL1, Gal-9, CD4, CD20, CD68, CD86, D2-40, and CD31. In certain embodiments, the immunophenotype is determined using immunocytochemistry, immunoblotting, flow cytometry, or fluorescence-activated cell sorting. The biological sample is preferably an endomyocardial biopsy (EMB) sample. In some subjects, the heart transplant rejection is clinically silent rejection. In certain aspects of the invention, the methods further comprise administering one or more immunosuppressive drugs to impede or inhibit the rejection. In certain aspects, the diagnosing, predicting, or monitoring transplant status or outcome comprises determining, modifying, or maintaining an immunosuppressive regimen based on modulation therapeutic targets differentially expressed in the EMB.

In another aspect of the invention, a method for identifying cardiac transplant tissue rejection in a human subject is provided. An exemplary method comprises determining a first immunophenotype profile in an EMB sample taken from said human subject, wherein said first immunophenotype profile comprises the protein expression levels of CD3, CD8, FoxP3, IL17, PDL1, Gal-9, CD4, CD20, CD68, CD86, D2-40, and CD31; and comparing said first immunophenotype profile to a second immunophenotype profile, wherein said second immunophenotype profile comprises the protein expression levels of CD3, CD8, FoxP3, IL17, PDL1, Gal-9, CD4, CD20, CD68, CD86, D2-40, and CD31 obtained from EMB samples collected from a human cardiac transplant population that does not have cardiac transplant tissue rejection, wherein a statistically significant alteration in proportional expression and intensity distribution patterns of CD3, CD8, FoxP3, IL17, PDL1, Gal-9, CD4, CD20, CD68, CD86, D2-40, and CD31 in said first immunophenotype profile compared to said second immunophenotype profile is indicative of cardiac transplant tissue rejection in said human subject.

The invention also provides a method for identifying a subject at risk for future cardiac transplant tissue rejection, comprising: determining a first immunophenotype profile in an EMB sample taken from said human subject, wherein said first immunophenotype profile comprises the protein expression levels of CD3, CD8, FoxP3, IL17, PDL1, Gal-9, CD4, CD20, CD68, CD86, D2-40, and CD31; and comparing said first immunophenotype profile to a second immunophenotype profile, wherein said second immunophenotype profile comprises the protein expression levels of CD3, CD8, FoxP3, IL17, PDL1, Gal-9, CD4, CD20, CD68, CD86, D2-40, and CD31 obtained from EMB samples collected from a human cardiac transplant population that does not have cardiac transplant tissue rejection, wherein a statistically significant alteration in proportional expression and intensity distribution patterns distributions of CD3, CD8, FoxP3, IL17, PDL1, Gal-9, CD4, CD20, CD68, CD86, D2-40, and CD31 in said first immunophenotype profile compared to said second immunophenotype profile is indicative of increased risk of future cardiac transplant tissue rejection in said human subject.

In yet another embodiment of the invention, a computer-implemented method for detection of an increased risk of cardiac transplant rejection in a subject in need thereof is disclosed. An exemplary method entails executing on a processor the steps of: performing quantitative pattern analysis of immunofluorescence data corresponding to an immunophenotype in an EMB indicative of undesirable immune cell infiltration correlated with allograft rejection to determine a level of spatial heterogeneity or similarity with an immunophenotype in standard subject not experiencing allograft rejection; and assigning an allograft rejection risk based on the level of spatial heterogeneity or similarity determined during said performing step. In certain aspects of the method, the immunophenotype is assigned based on differential proportional expression levels of immune cell markers in said EMB, indicative of future transplant rejection risk. In preferred embodiments, the immune cell and effector markers comprise at least three of CD3, CD8, FoxP3, IL17, PD-L1, Gal-9, CD4, CD20, CD68, CD86, D2-40, and CD31. Three preferred markers are at least PD-L1, FoxP3 and CD68. In other embodiments, expression levels of each of CD3, CD8, FoxP3, IL17, PD-L1, Gal-9, CD4, CD20, CD68, CD86, D2-40, and CD31 are determined. The method can further comprise mapping of cellular spatial and patterns, thereby characterizing in-situ interactions of immune cells and effectors. In another aspect, the invention method comprises performance of spatial analysis of lymphocyte location based on compartment segmentation, and assessment of interactions with myocytes via proximity graphs.

### BRIEF DESCRIPTION OF THE DRAWINGS

FIG. 1: Flow Diagram of Study Cohort, With Subgroups. CAR=cardiac allograft rejection; EMB=endomyocardial biopsy; ISHLT=International Society for Heart and Lung Transplantation; QC=quality control.

FIGS. 2A-2B: FIG. 2A) QmIF results for CD3+ and CD8+ cells, grouped by ISHLT grade and sub-grouped based on concordance or discordance between ISHLT grade and clinical evidence of rejection (see Table 1). Proportions of CD3+ and CD8+ cells correlate well with ISHLT grade, and do not help discriminate between cases classified by clinical rejection trajectory. FIG. 2B) QmIF results for CD68+, FoxP3+, and PD-L1+ cells, grouped by ISHLT grade and sub-grouped based on concordance or discordance between ISHLT grade and clinical evidence of rejection (Table 1). Within low ISHLT grades, there are significant differences between cases with clinically silent vs. evident rejection (CD68+: 0.99% vs. 0.14% [p<0.001], FoxP3+: 3.68% vs. 1.60% [p<0.001], PD-L1+: 7.59 vs. 1.3 [p<0.001]). Within the high ISHLT grades, there are also significant differences between silent and evident rejection (CD68+: 5.45% vs. 1.23% [p<0.001], FoxP3+: 3.55% vs. 1.88% [p<0.001], PD-L1+: 8.85 vs. 1.64 [p<0.001]). Overall, these QmIF markers correlate much better with clinical rejection severity then they do with traditional ISHLT grade, and may provide additional diagnostic value for assessing rejection on EMBs. Error bars denote standard errors.

FIGS. 3A-3D: H&E stained (FIGS. 3 A1, B1, C1, D1), 7-IF marker multiplex composite (FIGS. 3 A2, B2, C2, D2), and selected single IF-marker (FIGS. 3 A3-6, B3-6, C3-6, D3-6) endomyocardial biopsy (EMB) slides. Panels in FIG. 3A are a representative ‘concordant low ISHLT grade’ EMB with low ISHLT grade and no clinical evidence of allograft injury. Panels in FIG. 3B are from a ‘discordant low ISHLT grade’ EMB, with low histologic grade but clinically evident allograft injury. Panels in FIG. 3C are from a ‘disconcordant high ISHLT grade’ EMB, and panels in FIG. 3D are from a ‘concordant high ISHLT grade’ EMB. While rough estimates of basophilic cellular infiltrates due not appear markedly different within grade in H&E slides (FIGS. 3 A1 vs. B1, and C1 vs. D1), multiplex IF profiles within grade visibly differ between cases with and without evident allograft injury. Note diffuse PD-L1 (green) staining within myocardium of patients with clinically silent CAR (A5, C5), in comparison to clinically evident CAR (B5, D5). Also note profound PD-L1 and CD68 within cellular infiltrate in discordant high grade EMB (C4), as well as the higher proportion (albeit overall low density) of FoxP3 cells when compared to the concordant high grade EMB (C6 vs. D6).

FIG. 4: QmIF results for CD68+, FoxP3+, and PD-L1+ cells, grouped by future cardiac allograft rejection (CAR) risk and temporal proximity to a high-grade, clinically evident rejection event. Never-CAR cases have markedly higher proportions of immune-modulating FoxP3 and PD-L1 than future-CAR cases [FoxP3: 10.82% vs. 1.32% (p<0.001), PD-L1: 10.7% vs. 1.92% (p<0.001)]. Future-CAR cases when assessed by temporal proximity to a serious rejection event demonstrate an almost complete loss of detectable IF signal for each of these potentially allograft-protective markers by 3-6 weeks prior to serious rejection [FoxP3: 3.07% vs. 0% (p<0.001), PD-L1: 3.19% vs. 0.67% (p<0.001)]. Error bars=standard error.

FIGS. 5A-5B: Graphical representation of diagnostic and predictive potential. FIG. 5A: Biplot of 18 features from pilot QmIF experiment on n=33 cases of either clinically silent (red) or clinically evident (green) rejection events. Clear segregation between the groups is visible. FIG. 5B: biplot 8 features from the pilot QmIF experiment described in D.3, deployed on n=17 FR and NR patients.

FIG. 6: A summary slide showing that quantitative multiplexed immune-phenotyping of cardiac allograft biopsies provides novel diagnostic and prognostic information about allograft health. Reduced proportions of cells expressing PD-L1 and FoxP3 are associated with clinical evidence of serious allograft injury even when conventional histologic analysis provides falsely reassuring histologic rejection grades. The proportions of PD-L1- and FoxP3-expressing cells are dynamic within cardiac allografts, and reduced levels often precede future rejection.

### DETAILED DESCRIPTION OF THE INVENTION

Deep-phenotyping of allo-immune responses in EMB tissue provides an opportunity for more personalized diagnosis and risk stratification and a means of identifying the most biologically important mechanisms for therapeutic targeting. The ISHLT has acknowledged the need for “further characterization of the nature of the inflammatory infiltrate” in order to extract additional clinically relevant information from EMB samples 7, but despite this 15-year old call to action, there has been very limited application of tissue-level immune phenotyping in human heart transplant tissues.

Recognizing that guideline-directed histologic grading of endomyocardial biopsy tissue samples for rejection surveillance has limited diagnostic accuracy, quantitative, in situ characterization was performed of several important immune cell types in a retrospective cohort of clinical endomyocardial tissue samples. Using a multiplexed immunofluorescence immune-phenotyping panel along with computer image analysis techniques, we performed fully quantitative in-situ characterization of several important immune cells and effectors (CD3, CD8, CD68, FoxP3, and PD-L1) within formalin-fixed/paraffin embedded, clinical endomyocardial tissue samples. A carefully constructed cohort permits assessments of immune phenotype differences between tissues with low vs. high conventional histologic grades, between tissues representing clinically silent vs. clinically evident rejection trajectories, and between tissues from patients who will go on to experience serious rejection (future-rejectors) vs. those who will not (never-rejectors).

The results show that biopsies with high histologic grades, regardless of clinical trajectory, exhibited greater proportions of CD3+ and CD8+ cells than biopsies with low histologic grades [CD3+: 12.5±0.1% vs. 3.3±0.04% (p<0.001), CD8+: 7.55±0.08% vs. 1.16±0.04% (p<0.001)]. Biopsies associated with clinically silent trajectories, regardless of histologic grade, had greater proportions of CD68+, FoxP3+, and PD-L1+ cells than biopsies associated with clinically evident allograft injury [CD68+: 1.91±0.04% vs. 1.05±0.03% (p<0.001), FoxP3+: 3.61±0.26% vs. 1.86±0.83% (p<0.001), PD-L1+: 7.86±0.09% vs. 1.58±0.04% (p<0.001)]. PD-L1+ and FoxP3+ cell proportions were also greater in biopsies from ‘never-rejector’ patients as compared to ‘future-rejector’ patients [PD-L1: 10.7±0.1% vs. 1.92±0.04% (p<0.001), FoxP3+: 10.82±1.3% vs. 1.32±0.27% (p<0.001)], with ‘future-rejectors’ demonstrating a dramatic loss of PD-L1+ immunofluorescent signal in the 3-6 weeks preceding serious rejection.

The findings demonstrated herein provide new avenues for patient-specific tailoring of rejection surveillance and highlight the diagnostic, prognostic, and therapeutic roles played by regulatory T cells and the checkpoint system in organ transplantation survival and rejection. The proportion of PD-L1+, FoxP3+, and CD68+ cells in cardiac allografts has been identified in an immunophenotype which is predictive of immediate and future risk of serious allograft injury.

### Definitions

For convenience, certain terms employed herein, in the specification, examples and appended claims are collected here.

The term “transplant rejection” encompasses both acute and chronic transplant rejection. “Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or CR” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The phrase “expression profile” refers to the results obtained upon differentially determining expression of at least 3, at least 4, or at least 5, at least 6, at least 7, at least 8, at least 9 or at least 10 genes or their encoded protein products, when compared to a standard. The profile is assigned to a given subject, which reflects comparative results between his or her expression of the at least 3, at least 4, or at least 5, at least 6, at least 7, at least 8, at least 9 or at least 10 genes or their products as compared to a standard. In one embodiment, the expression profile further comprises a determination of relative expression of nucleic acids, which do not code for a functional protein, as compared to the standard.

The term “differentially expressed” refers to a relative abundance or absence of expression in a subject as compared to a standard. Differential expression refers to changed expression, either higher or lower, in the subject, as compared to the standard.

Differential gene expression may include in one embodiment, a comparison of expression between two or more genes or a comparison of two differently processed products of the same gene, which differ between control subjects and subjects in which transplant was rejected, or in another embodiment, in the same subject pre- and post transplantation. Differential expression refers in one embodiment to quantitative, as well as in another embodiment, qualitative, differences in the temporal or cellular expression pattern in a gene or its protein expression products as described herein.

In certain embodiments, a gene expression profile is compiled using a tissue biopsy for evaluation, or in another embodiment, the end-stage diseased organ, whose replacement is desired is used as the source for gene expression profile. In other embodiments, a sample of peripheral blood of a subject being evaluated.

A protein or gene expression profile compiled using the methods described herein will comprise proteins differentially expressed in successful transplant recipients, as compared to those prone to transplant rejection. The pattern of the differentially expressed proteins will comprise increased expression of certain proteins simultaneous with diminished expression of at other proteins, in subjects more likely to reject a transplant, whereas the reverse profile is more predictive of success of a transplant in a given subject.

In another embodiment, the expression profile is a relative value as compared to a standard. In one embodiment the term “standard” may refer to a pooled sample of successful recipients for the same organ transplant. In another embodiment, standard may be ethnically- or gender- or age-matched recipients. It is to be understood that the standard may be derived from any subject, or pool of subjects, whose expression profile or profiles, once generated, is sufficient to detect even minute relative differences in expression, when compared to a potential transplant recipient, or in another embodiment, transplant recipient.

In one embodiment, “increased expression” refers to an increase in the level or in another embodiment, activity of target gene product relative to the level or activity of target gene product in a standard. In another embodiment, increased expression refers to between a 10 to about a 250% increase in mRNA levels, or in another embodiment, in protein levels. In another embodiment, increased expression refers to changes in gene expression at the mRNA or protein level, in terms of its pattern of expression in particular examples, such as, for example, and in one embodiment, increased expression in tissue, but not in the blood, for example, in damaged tissue for which the transplant is required. In one embodiment, increased expression is synonymous with overexpression, or stimulated expression. In another embodiment, increased expression is a relative determination, wherein expression is greater than the standard, or in cases where expression is absent in the standard, this despite expression being barely detectable in the subject. It is to be understood that any such circumstance described hereinabove, represents increased expression for the methods of this invention.

In one embodiment, “diminished expression” refers to a reduction in the level or in another embodiment, activity of target gene product relative to the level or activity of the target gene product in a standard. In one embodiment, diminished expression is synonymous with decreased expression, or in another embodiment with under expression. In one embodiment, the expression of the gene or product is absent in the subject, or slightly less than the standard. In one embodiment, the expression of the gene product is diminished by at least 25%

In one embodiment, “compared to a standard”, refers to relative changes in expression where the standard is derived from a single individual, or is derived from pooled subjects who have successfully undergone a transplant. In another embodiment, a standard can be derived from a single subject following about 1 to about 5 years of having undergone successful transplantion. In one embodiment, a standard can be derived from a subject who has undergone transplant of the specific tissue for which the subject is being evaluated, such as, for example, being obtained from a subject having undergone a successful cardiac transplant. In another embodiment, the standard is derived from a subject who has undergone transplant of a different tissue type than that sought by the recipient, however, the two individuals, or pool of individuals are of a similar genetic background.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a human. A subject can be male or female.

A subject can be one who has been previously diagnosed with or identified as suffering from or having a condition in need of treatment (e.g. a subject undergoing an allograft or having an autoimmune disease) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat” “treatment” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

The following methods are provided to facilitate the practice of Example I.

Cohort Considerations. The study cohort was selected from the transplant records at the Hospital of the University of Pennsylvania, and consisted of biopsy events that occurred between 2007 and 2013. Cases were manually selected to allow for exploration of the potential diagnostic and prognostic implications of performing immune-phenotyping within EMB tissue. Specifically, the cohort of N=46 transplant EMBs described in this manuscript was selected to permit assessments of how immune cell populations differ between tissues with low vs. high ISHLT grades, between tissues corresponding to clinically silent vs. clinically evident rejection trajectories, and between tissues from patients who will go on to experience serious rejection (future-rejection) vs. those who will not (never-rejection). The retrospective chart review and analysis of archived tissue specimens employed in this research were approved by the Institutional Review Board at the University of Pennsylvania.

For these retrospective cases, the ISHLT histologic grade assigned by the attending pathologist at the time of the EMB procedure was used as the reference standard for rejection diagnosis. These grades were further simplified by assigning a binary histologic grade label: “low grade” rejection was defined as ISHLT 2004 consensus criteria histologic grades 0R or 1R and “high grade” rejection was defined as ISHLT histologic grade 2R or 3R. This grouping typically defines the distinction used to determine whether or not augmented immunosuppressive therapy is prescribed.³⁴

For the same 46 EMB events, the distinction between “clinically silent” and “clinically evident” rejection was made based on a set of major and minor criteria to determine whether allograft injury was present. Clinical metadata from within seven days of each EMB event were collected to allow for determination of clinical trajectory. These data were derived from electronic health record (EHR)-documented symptoms, physical exam findings, lab results, echocardiographic parameters, EKG findings, and invasive hemodynamic data. The major criteria in Table 1 for differentiating ‘clinically evident’ from ‘clinically silent’ rejection trajectories are based on definitions of ‘hemodynamic compromise’ in previous prospective investigations of allograft rejection³⁵⁻³⁷, and provide high specificity for clinically significant rejection. The minor criteria in Table 1A permit identification of clinically significant cases with greater sensitivity. By design, a subset of each clinical trajectory category represented an EMB with clinical-histologic discordance (low histologic grade meeting criteria for clinically evident rejection, or high histologic grade with none of the criteria met for clinically evident rejection). All determinations of clinically evident vs. clinically silent rejection were completed prior to the performance of immunostaining.

Finally, the 19 low ISHLT grade, clinically silent EMBs in this cohort were further classified by the patient-level incidence of future serious CAR events. These EMBs were assigned a binary label as either ‘future-rejection’ or ‘never-rejection’, based on whether a serious CAR event occurred within the first 3-years post-transplant. These cases allow for assessment of whether meaningful differences occur in IF markers in advance of a serious CAR event.

TABLE 1A
Criteria for determining ‘clinically evident’ rejection.
Admission to hospital for rejection treatment, along with 1 major OR 2 minor criteria:

MAJOR CRITERIA:
Cardiac index (CI) ≤2.0 and use of inotropes
Absolute decrease in left ventricular ejection fraction (LVEF) of ≥20%
MINOR CRITERIA:
CI ≤2.3, provided this represents a ≥20% decrease in CI from baseline
Right atrial pressure >10 mmHg OR pulmonary capillary wedge pressure >18 mmHg
provided this represents a ≥40% increase from baseline
Absolute decrease in LVEF of ≥10% AND to a level of ≤50%
NEW arrhythmia - atrial fibrillation, flutter, or ventricular arrhythmia
NEW low voltage EKG not due to pericardial effusion or pulmonary disease
Cardiac troponin elevated ≥3× the upper limit of normal and ≥3× the patients baseline,
NOT due to coronary artery disease/graft vasculopathy
DOCUMENTED diagnosis of increased left ventricular wall thickness AND an LV wall
thickness increase of >2 mm from baseline value
DOCUMENTED new or worsened right ventricular dysfunction by echo
DOCUMENTED clinical signs or symptoms of rejection or heart failure:
Signs = NEW gallop, NEW low pulse volumes, NEW Rales.
Symptoms = NEW or worsened dyspnea, orthopnea, exercise intolerance documented by
provider as likely due to cardiac cause.

### Retrospective Sample Acquisition and Preparation.

All EMB tissues analyzed for this study had been sampled using a standard percutaneous method, fixed in 4% paraformaldehyde, and embedded in paraffin wax as per usual post-transplant clinical care and pathology laboratory workflows at the Hospital of the University of Pennsylvania. Five, 4 m thick serial sections were cut from formalin-fixed paraffin embedded (FFPE) blocks and mounted onto positively-charged glass microscope slides (48382-119, VWR, Radnor, PA) used for immunohistochemistry, with one section per slide. All cut, unstained tissue sections were stored in a nitrogen chamber to minimize oxidation and degradation of tissue epitopes. In addition to EMB slides, two slides from native (non-transplant) heart tissue obtained from cadaveric organ donors and one slide from human lymph node tissue were used as ‘negative’ and ‘positive’ staining controls, respectively.

### Target Selection for Multiplex Immunofluorescence.

The cytotoxic T cell marker CD8 was selected due to recognition that this cell population is a primary effector of myocyte injury during cellular rejection¹⁸⁻²⁰. Regulatory T cell transcription factor FoxP3 was selected due to the immune-modulatory, anti-inflammatory effects these IL-10 and TGF-β secreting cells are thought to exert in renal allografts²¹⁻²³ and animal models of heart transplantation^{24, 25}. Monocyte lineage marker CD68 is used in the diagnosis of antibody mediated rejection, but also has also been implicated in cellular rejection, albeit with conflicting results on the effects they exert^{26, 27}. Finally, PD-L1 (programmed death ligand) is one component of the PD-L1/PD1 immune “checkpoint’ molecules that interact to suppress cytotoxic actions of activated T-cells. Though checkpoint inhibitors used in cancer immunotherapy have been implicated in myocarditis, a role for checkpoint molecules in human heart transplantation has included only case reports of severe rejection following oncologic PD1/PD-L1 inhibitor treatment²⁸⁻³⁰. However, the checkpoint pathway has been recognized for its role in abrogating T-cell responses and promoting tolerance in animal models of organ transplantation³¹⁻³³.

### Quantitative Multiplex Immunofluorescence Methods:

All study tissue samples were assigned de-identified study IDs and sent to Akoya Biosciences (Hopkinton, MA) for multiplex staining, slide digitization, and per-slide image quantification. The scientific team performing staining and image quantification was blinded to patient outcomes. Details of deparaffinization, reagent preparation, and automated staining workflows are described below. Details on antibodies utilized for multiplex staining are summarized in Table 1B.

TABLE 1B
Antibodies used for QmIF

| Marker | Reference ID | Company | Species |
|---|---|---|---|
| CD3 | ab16669 | Abcam, Cambridge, MA | Rabbit |
| CD8 | PA0183 | Leica | Mouse |
| CD68 | M087629-2 | Agilent, Santa Clara, CA | Mouse |
| FoxP3 | ab20034 | Abcam, Cambridge, MA | Mouse |
| PD-L1 | 13684 | Cell Signaling | Rabbit |
| | | Technologies, Danvers, | |
| | | MA | |
| Membrane marker | ab76020 | Abcam | Rabbit |
| cocktail (ATPase, | API 3116 AA | Biocare Medical | Mouse |
| CD33, CD45LcA) | M070101-2 | Agilent | Mouse |

Whole Slide Multispectral Image Acquisition: High-throughput whole slide multispectral (MOTiF) image scans were acquired on the Vectra Polaris (Akoya Biosciences) at 20×using narrow, multiband filters. Exposure times were set to avoid saturation.

### Image Quality Control and Annotation Selection:

Following image acquisition, scans were imported into Phenochart (Akoya Biosciences) to assess the image quality. Up to five 1×1 regions of interest (ROI) were annotated for each sample to conduct quantitative image analysis. Some samples were unable to yield five ROI due to confounding factors, such as tissue folds, small tissue biopsy size, or large areas without myocardium (e.g. vessels, fibrosis, fat).

### Quantitative Multispectral Image Analysis:

A spectral library was created from the stained library slides, and the autofluorescence spectrum was isolated using the autofluorescence slide. All annotated ROIs were imported into a new project in inForm Tissue Finder (Akoya Biosciences) and were spectrally unmixed using the generated spectral library. Of note, the image analyst was blinded to the slide cohort categories to avoid bias into the analysis.

### Staining Quality Control:

Individual fluorophore signal intensities, in normalized counts, were assessed on the multiplex imagery following spectral unmixing to confirm proper staining intensity levels and absence of residual cross talk.

### InForm Analysis Workflow:

The Trainable Tissue Segmentation module was used to automatically segment all samples into total cardiac tissue and background based on hand drawn training regions. Cell segmentation was conducted using the Adaptive Cell Segmentation, which uses algorithms to account for variations in staining and background levels within and across images to identify cellular compartments from multiple image planes. The algorithm first identifies the cell nuclei (via DAPI staining), followed by the cell membrane and cytoplasm for each, and has further settings for cell splitting and staining quality. The Phenotyping module, which utilizes a user-trainable random forest classifier, learned and phenotyped all markers, except for PD-L1, in which the staining intensity was scored per-cell on a 0-3+ scale using thresholding to enable H-score calculations (see statistical methods below). All cell segmentation and scoring data and imagery were exported for further analysis.

### Statistical Methods.

Raw cell count (and PD-L1 intensity) data generated by inForm quantitative image analysis were sent to the University of Pennsylvania for further analysis. Cases were analyzed by pre-specified groups as described in ‘Cohort Considerations.’ For CD3+, CD8+, and CD68+, and PD-L1 cell count data, values were normalized by total cell counts to account for differences in tissue size and myocardial area. FoxP3+ cells were normalized by the total T-cell (CD3+) cell count. Group-wise chi-squared testing was performed on the normalized cell count data. Comparisons included cases grouped by high vs low ISHLT histologic grade, by ‘clinically evident’ vs ‘clinically silent’ rejection determination, and by patient-level ‘future-rejection’ vs. ‘never-rejection’ label. Cases were also analyzed by contingency table designation, as it pertains to the concordance between high/low ISHLT grade and the presence or absence of clinically evident allograft injury (eg. ‘concordant-positive’=high ISHLT grade/clinically evident CAR, ‘concordant-negative’=low ISHLT grade/clinically silent CAR, ‘discordant-positive’=high ISHLT grade/clinically silent CAR, and ‘discordant-negative’=low ISHLT grade/clinically evident CAR).

Semi-quantitative analysis of PD-L1 staining intensity was also performed, with PD-L1+ status placed into one of four categories based on intensity (level 0=no PD-L1, level 1=low intensity, level 2=moderate intensity, level 3=high intensity. PD-L1 Histology scores (H-Scores) based on a method described previously³⁸. Briefly, the total number of cells and the percent of cells in each PD-L1 staining level were counted in three randomly selected fields under high magnification. PD-L1 H score was then calculated for each case according to the following formula: H score=0*% of level 0 cells+1*% of level 1 intensity cells+2*% of level 2 intensity cells+3*% of level 3 intensity cells. Because H-Scores were tabulated on a per-patient basis rather than a per-cell basis, a per-patient analysis of this metric was performed using a Wilcoxian Rank-Sum test to assess differences between pre-specified study groups. All statistical analysis was performed in Stata 15 (StataCorp, 2015), and p-values<0.05 were considered significant for all analyses.

The following examples are provided to illustrate certain embodiments of the invention. They are not intended to limit the invention in any way.

### Example I

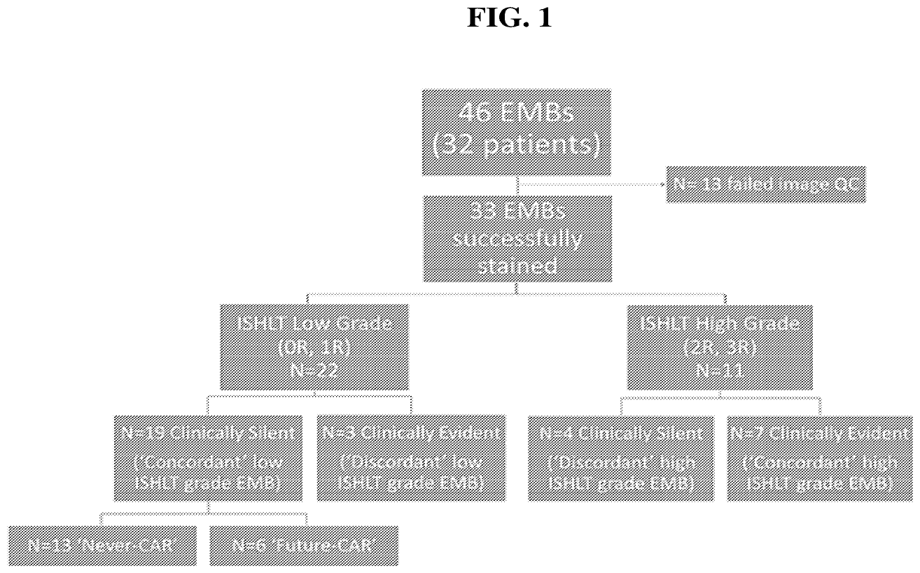

A total of 46 EMB episodes plus three additional control tissues were selected for this analysis, and their characteristics are detailed in Table 1C. In total, 36/49 (74%) of the formalin-fixed paraffin-embedded tissue slides (including the n=3 non-EMB controls) submitted for QmIF underwent successful staining, quality control assessment, and comprehensive quantitative analysis. Of the 33 transplant EMB cases that completed study staining and analysis (see FIG. 1), 22 had low ISHLT grades (1R and 0R) and 11 had high ISHLT grades (2R and 3R). Of the cases with low ISHLT grades, 19 had clinically silent rejection and three had clinically evident rejection. Of the cases with high ISHLT grades, seven had clinically evident rejection and four had clinically silent rejection. None of the instances of clinically evident rejections corresponding to low ISHLT grades met ISHLT criteria for antibody mediated rejection.³⁹ Specifically, none had new donor specific antibodies or significant C4d deposition by clinical immunofluorescence staining. Of the 19 cases with low ISHLT grades and clinically silent rejection, 6 would go on to have a future rejection within ˜1 year and 13 would never reject over the next three years. Of the four discordant high ISHLT grade EMBs associated with a clinically silent course, none manifested evidence of clinically evident allograft rejection in the subsequent three months.

TABLE 1C
Experimental cohort by pre-specified subgroup
and QmIF quality control (QC) result.

| | Biopsy | Histologic | Clinical | Future CAR | | |
|---|---|---|---|---|---|---|
| Patient | Event | Grade | Trajectory | Risk | Time point | QC |
| 1 | A | High | Evident Rejection | | | Fail |
| | B | Low | Silent Rejection | Future-CAR | <6 wks before 1A | Pass |
| | C | Low | Silent Rejection | Future-CAR | >6 mo before 1A | Fail |
| 2 | A | High | Evident Rejection | | | Pass |
| | B | Low | Silent Rejection | Future-CAR | <6 wks before 2A | Pass |
| | C | Low | Silent Rejection | Future-CAR | >6 mo before 2A | Pass |
| 3 | A | High | Evident Rejection | | | Pass |
| | B | Low | Silent Rejection | Future-CAR | <6 wks before 3A | Fail |
| | C | Low | Silent Rejection | Future-CAR | >6 mo before 3A | Fail |
| 4 | A | High | Evident Rejection | | | Pass |
| | B | Low | Silent Rejection | Future-CAR | <6 wks before 4A | Pass |
| | C | Low | Silent Rejection | Future-CAR | >6 mo before 4A | Pass |
| 5 | A | High | Evident Rejection | | | Pass |
| | B | Low | Silent Rejection | Future-CAR | <6 wks before 5A | Fail |
| | C | Low | Silent Rejection | Future-CAR | >6 mo before 5A | Pass |
| 6 | A | Low | Silent Rejection | Never-CAR | | Fail |
| | B | Low | Silent Rejection | Never-CAR | >6 mo before 6A | Pass |
| 7 | A | Low | Silent Rejection | Never-CAR | | Pass |
| | B | Low | Silent Rejection | Never-CAR | >6 mo before 7A | Fail |
| 8 | A | Low | Silent Rejection | Never-CAR | | Pass |
| | B | Low | Silent Rejection | Never-CAR | >6 mo before 8A | Fail |
| 9 | A | Low | Silent Rejection | Never-CAR | | Pass |
| | B | Low | Silent Rejection | Never-CAR | >6 mo before 9A | Pass |
| 10 | A | Low | Silent Rejection | Never-CAR | | Pass |
| 11 | A | Low | Silent Rejection | Never-CAR | | Pass |
| 12 | A | Low | Silent Rejection | Never-CAR | | Pass |
| 13 | A | Low | Silent Rejection | Never-CAR | | Pass |
| 14 | A | Low | Silent Rejection | Never-CAR | | Pass |
| 15 | A | Low | Silent Rejection | Never-CAR | | Pass |
| 16 | A | High | Evident Rejection | | | Pass |
| 17 | A | High | Evident Rejection | | | Fail |
| 18 | A | High | Evident Rejection | | | Pass |
| 19 | A | High | Evident Rejection | | | Pass |
| 20 | A | High | Evident Rejection | | | Fail |
| 21 | A | Low | Evident Rejection | | | Pass |
| 22 | A | Low | Evident Rejection | | | Pass |
| 23 | A | Low | Evident Rejection | | | Pass |
| 24 | A | Low | Evident Rejection | | | Fail |
| 25 | A | Low | Evident Rejection | | | Fail |
| 26 | A | High | Silent Rejection | | | Pass |
| 27 | A | High | Silent Rejection | | | Fail |
| 28 | A | High | Silent Rejection | | | Pass |
| 29 | A | High | Silent Rejection | | | Pass |
| 30 | A | High | Silent Rejection | | | Pass |
| 31 | A | Low | Silent Rejection | Never-CAR | | Pass |
| 32 | A | Low | Silent Rejection | Never-CAR | | Pass |

| | |
|---|---|
| Heart Control 1 | Pass |
| Heart control 2 | Pass |
| Lymph Node | Pass |

### Tissue Immunophenotyping Results.

For the 33 EMB cases that completed QmIF staining, there were a total of 191,331 cells in the regions that underwent quantitative analysis, with CD3+ cells representing the most common immune cell type, as summarized in Table 2A.

TABLE 2A
QmIF total cell count results

| Cell type | Cell count | % of Total |
|---|---|---|
| CD8+ | 8646 | 4.52% |
| CD3+ | 15571 | 8.14% |
| CD68+ | 2814 | 1.47% |
| FoxP3+ | 382 | 0.20% |
| PDL1+ | 8932 | 4.67% |
| DAPI (Total Cells) | 191331 | 100.00% |

Table 2B shows the study results grouped by ISHLT grade (high vs. low) and by clinical rejection syndrome (clinically silent vs. clinically evident). High grade EMBs have significantly higher proportions of CD3+ and CD8+ cells than low grade EMBs (p<0.001). Grade-agnostic grouping by rejection syndrome severity demonstrates analogous results, with serious, clinically evident rejection events having higher proportions of CD3+ and CD8+ cells than clinically silent rejection events (p<0.001).

TABLE 2B
QmIF results by immune cell type. Cases grouped by clinical
rejection trajectory and conventional histologic grade.

| | | | p- | Clinically | Clinically | p- |
|---|---|---|---|---|---|---|
| Marker | Low Grade | High Grade | value | Silent | Evident | value |
| CD8+ (% of total | 1.16 ± .04 | 7.55 ± .08 | <0.001 | 2.93 ± .05 | 6.06 ± .07 | <0.001 |
| cells ± SE) | (1049/90731) | (7597/100600) | | (2754/94098) | (5892/97233) | |
| CD3+ (% of total | 3.3 ± .06 | 12.5 ± .1 | <0.001 | 5.6 ± .07 | 10.6 ± .09 | <0.001 |
| cells ± SE) | (2995/90731) | (12576/100600) | | (5266/94098) | (10305/97233) | |
| CD68+ (% of total | 0.83 ± .03 | 2.04 ± .04 | <0.001 | 1.91 ± .04 | 1.05 ± .03 | <0.001 |
| cells ± SE) | (757/90731) | (2057/100600) | | (1797/94098) | (1017/97233) | |
| FoxP3+ (% of CD3+ | 3.21 ± .32 | 2.27 ± .13 | 0.039 | 3.61 ± .26 | 1.86 ± .13 | <0.001 |
| cells ± SE) | (96/2995) | (286/12576) | | (190/5266) | (192/10305) | |
| PD-L1+ (% of total | 6.48 ± .08 | 3.04 ± .05 | <0.001 | 7.86 ± .09 | 1.58 ± .04 | <0.001 |
| cells ± SE) | (5876/90731) | (3056/100600) | | (7392/94098) | (1540/97233) | |
| PD-L1 H-Score | 3 (1-16) | 4 (1-11) | 0.88 | 5 (2-16) | 1 (0-2) | 0.018 |
| (per-EMB median, | | | | | | |
| [IQR]) | | | | | | |

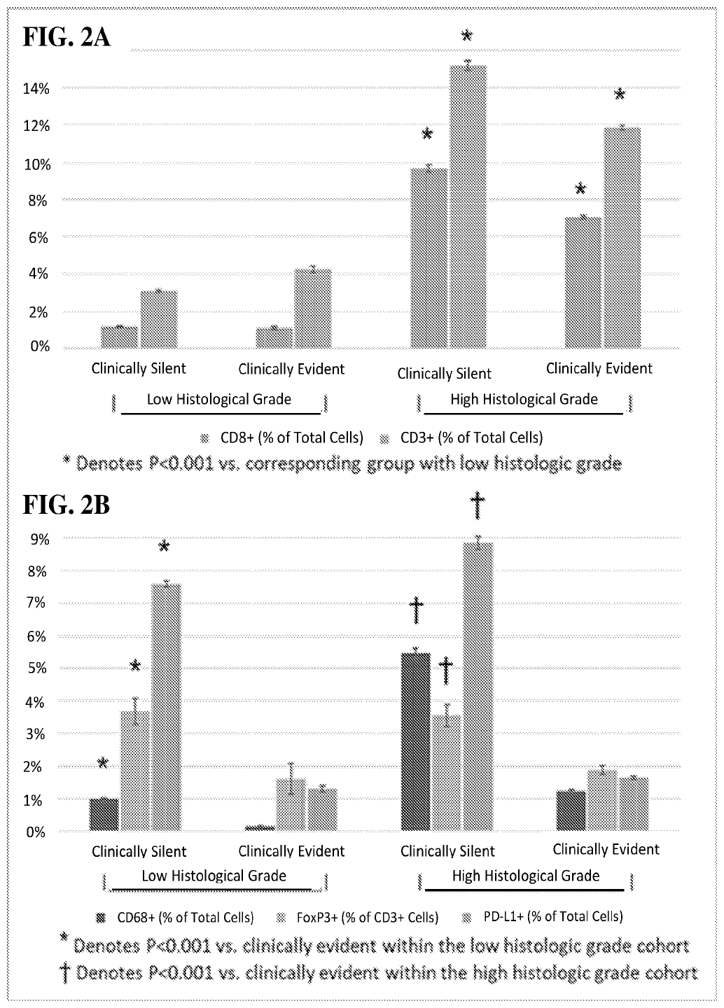

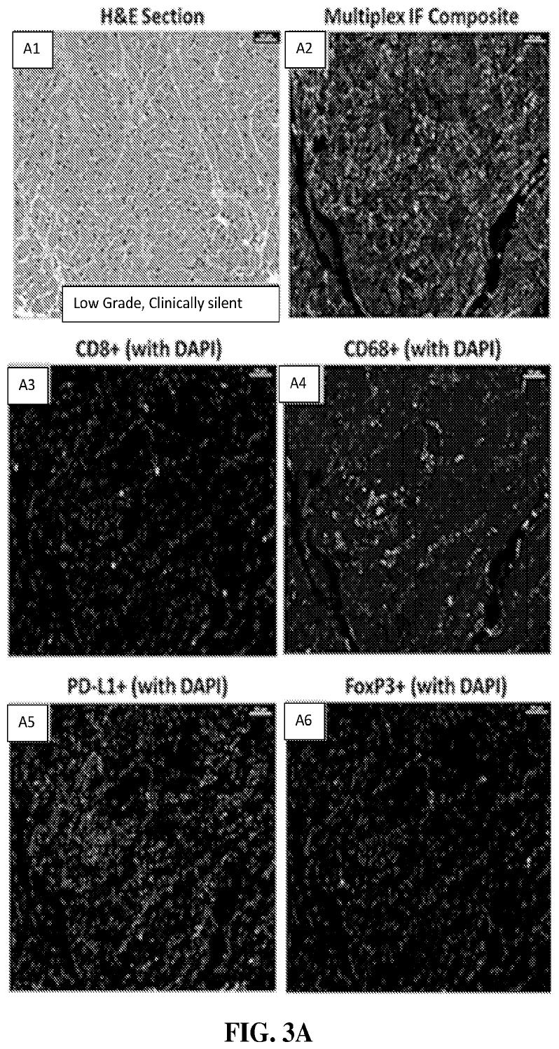

FIG. 2A provides a compelling visual demonstration of how these conventional T-cell markers correlate predominantly with grade classification, and fail to discriminate between cases with vs. without serious clinical rejection syndromes.

High grade EMBs also have significantly higher proportions of macrophage marker CD68 when compared to low grade EMBs (p<0.001), but unlike the traditional T-cell markers CD3 and CD8, the opposite relationship is seen when cases are grouped by the severity of the clinical rejection syndrome. Clinically silent rejection events have a significantly higher proportion of CD68+ cells than clinically evident rejection events (p<0.001). An examination of the results from EMBs with clinical-histologic discordance provides the explanation for these opposing results (Table 2C, FIG. 2B), with discordant high-grade cases manifesting by far the highest proportion of CD68+ cells (5.45%, p<0.001) while discordant low-grade cases manifest by far the lowest (0.14%, p<0.001).

TABLE 2C
QmIF results by immune cell type, with cases grouped by contingency table designation based
on concordance vs. discordance of clinical rejection severity and ISHLT histologic grade.

| | Contingency Table Groups | | | | P-values for Inter-Group Comparisons | | | | | |
|---|---|---|---|---|---|---|---|---|---|---|
| | Concord. | Discord. | Discord. | Concord. | C-Low | C-Low | C-Low | D-Low | D-Low | D-High |
| | Low Grade | High Grade | Low Grade | High Grade | vs | vs | vs | vs | vs | vs |
| Marker | (C-Low) | (D-High) | (D-Low) | (C-High) | D-Low | C-High | D-High | C-High | D-High | C-High |
| CD8+ (% of total | 1.17 ± .04 | 9.67 ± .09 | 1.1 ± .03 | 7.04 ± .08 | 0.5 | <0.001 | <0.001 | <0.001 | <0.001 | <0.001 |
| cells ± SE) | (871/74616) | (1883/19482) | (178/16115) | (5714/81118) | | | | | | |
| CD3+ (% of total | 3.09 ± .06 | 15.18 ± .11 | 4.26 ± .07 | 11.86 ± .1 | <0.001 | <0.001 | <0.001 | <0.001 | <0.001 | <0.001 |
| cells ± SE) | (2309/74616) | (2957 /19482) | (686/16115) | (9619/81118) | | | | | | |
| CD68+ (% of total | 0.99 ± .03 | 5.45 ± .07 | 0.14 ± .01 | 1.23 ± .03 | <0.001 | <0.001 | <0.001 | <0.001 | <0.001 | <0.001 |
| cells ± SE) | (735/74616) | (1062/19482) | (22/16115) | (995/81118) | | | | | | |
| FoxP3+ (% of CD3+ | 3.68 ± .39 | 3.55 ± .34 | 1.6 ± .48 | 1.88 ± .14 | <0.001 | <0.001 | 0.8 | 0.6 | <0.001 | <0.001 |
| cells ± SE) | (85/2309) | (105/2957) | (11/686) | (181/9619) | | | | | | |
| PD-L1+ (% of total | 7.59 ± .09 | 8.85 ± .09 | 1.3 ± .04 | 1.64 ± .04 | <0.001 | <0.001 | <0.001 | 0.002 | <0.001 | <0.001 |
| cells ± SE) | (5667/74516) | (1725/19482) | (209/16115) | (1331/81118) | | | | | | |
| PD-L1 H- | 4 (2-16) | 9 (6-19) | 1 (0-2) | 1 (0-4) | 0.09 | 0.13 | 0.24 | 0.64 | .034 | 0.037 |
| Score (per- | | | | | | | | | | |
| EMB median | | | | | | | | | | |
| [IQR]) | | | | | | | | | | |

Cells expressing the regulatory T cell marker FoxP3 are twice as abundant in clinically silent rejection events, as compared to clinically evident rejection both within the low histologic grade and the high histologic grade cohort (p<0.001 for both). Even more striking, PD-L1+ cells were more than four-fold more abundant in clinically silent cases compared with clinically evident cases, regardless of ISHLT grade. Table 2C and FIG. 2B highlight the strong, grade-independent correlation of FoxP3 and PD-L1, with similarly high proportions found either in low- or high-grade clinically silent EMBs and similarly low proportions found in either low- or high-grade clinically evident EMBs.

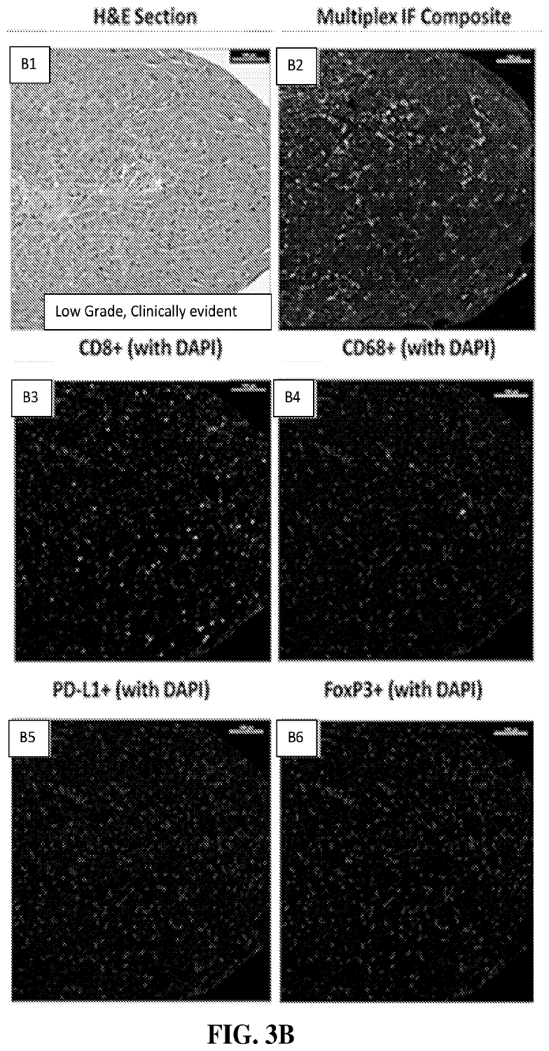

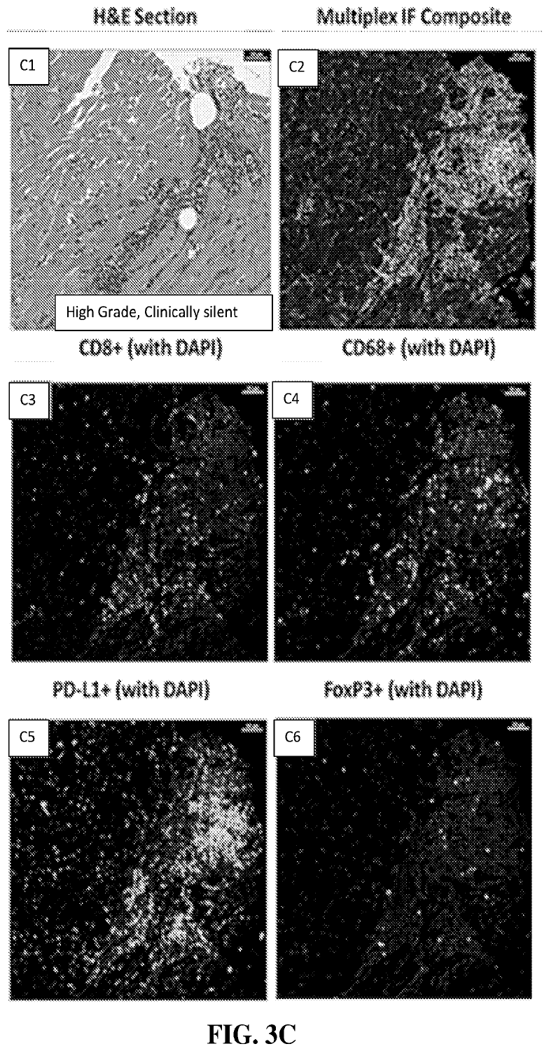

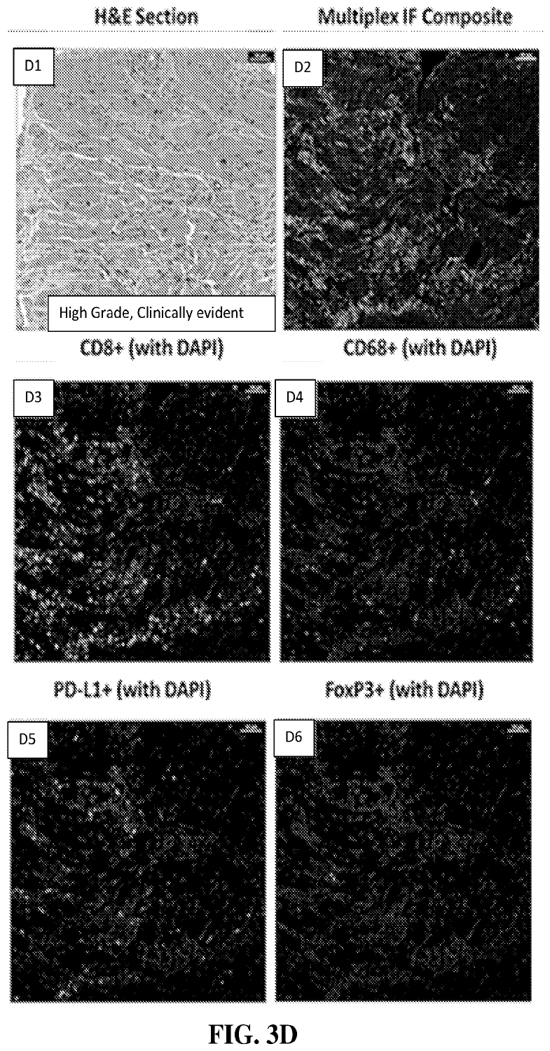

The per-patient PD-L1 H-score, a composite measure incorporating both PD-L1 staining prevalence and signal intensity, is strongly associated with clinical rejection syndrome but not with histologic grade (Table 2B, 2C, and FIG. 3B). Patients with clinically silent rejection syndromes, whether the EMB received a high or low grade, manifest PD-L1 H-scores that are at least 4-times higher than those seen for clinically evident rejection events of either grade class. Illustrative H&E and QmIF stained digital slides for high and low ISHLT grades with both concordant and discordant clinical rejection syndromes are shown in FIG. 3.

### Immunophenotyping ‘Future-Rejection’ Cases and ‘Never-Rejection’ Cases.

To assess whether immune phenotypes differ between patients who will suffer important rejection events in the future and those who will not, we analyzed the subset of cases in which serial, concordant, low-grade EMBs were available for individual patients. These patients were assigned a binary label as either ‘future-rejection’ or ‘never-rejection.’ For the pre-specified ‘future-rejection’ sub-group, six EMBs preceding a high-grade, clinically-evident rejection event successfully completed the QmIF analysis. This included three EMBs obtained >6 months prior to a rejection and three EMBs obtained 3-6 weeks prior to a rejection. For the ‘never-rejection’ group, there were 13 EMBs, but in only one instance was the same patient sampled twice (due to a high—and likely by-chance—rate of staining/image quality issues with the serial never-rejection subgroup). From the six ‘future-rejection’ EMB samples, a total of 26,371 cells were analyzed, while the 13 EMBs from the ‘never-rejection’ provided 48,245 cells for analysis.

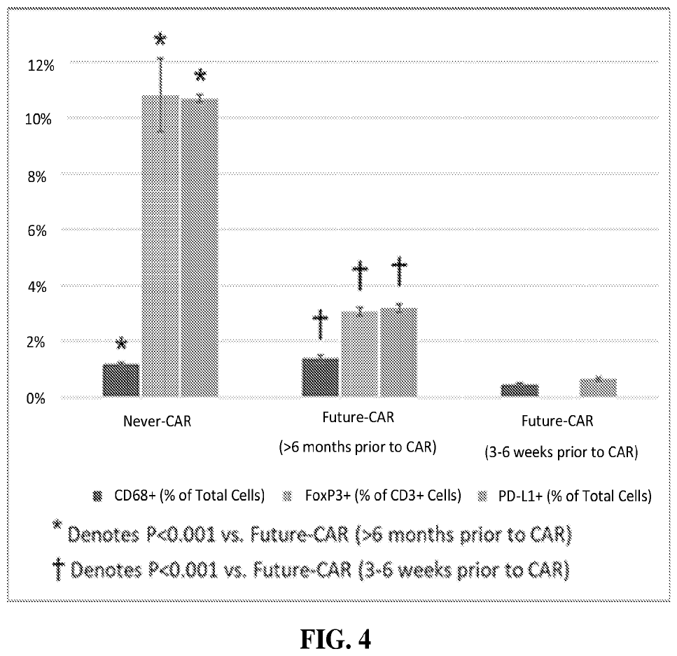

Despite relatively few cases, every QmIF marker displayed statistically significant differences between ‘future-rejection’ cases and ‘never-rejection’ cases, as shown in Table 3. Though these analyses were performed on biopsies that were uniformly of low histologic grade at a time when there was no evidence of allograft dysfunction or injury, the ‘future-rejection’ group more closely resembles the clinically-evident and high-grade rejection groups than it does the clinically silent or low-grade groups. When the ‘future-rejection’ group is analyzed by temporal proximity to the incident severe rejection event, an apparent progression emerges: compared to never-rejection cases, future-rejection cases at >6 months prior to rejection have a moderately reduced proportion of FoxP3+ cells, and PD-L1+ cells. By 3-6 weeks before clinically evident rejection, there is a dramatic and statistically significant drop-off to extremely low levels for FoxP3+, CD68+ and the PD-L1+ cells (Table 3 & FIG. 4).

TABLE 3
QmIF results for cases associated with future serious rejection
and those who never experience serious rejection.

| Marker | Never-Rejection | | Future-Rejection | | p-value |
|---|---|---|---|---|---|
| CD8+ (% of total cells ± SE) | 0.48 ± .02 | (230/48245) | 2.43 ± .05 | (641/26371) | <0.001 |
| CD3+ (% of total cells ± SE) | 1.19 ± .04 | (573/48245) | 6.58 ± .08 | (1736/26371) | <0.001 |
| CD68+ (% of total cells ± SE) | 1.01 ± .03 | (488/48245) | 0.94 ± .03 | (247/26371) | 0.32 |
| FoxP3+ (% of CD3+ cells ± SE) | 10.82 ± 1.3 | (62/573) | 1.32 ± .27 | (23/1736) | <0.001 |
| PD-L1+ (% of total cells ± SE) | 10.7 ± .1 | (5160/48245) | 1.92 ± .04 | (507/26371) | <0.001 |
| PD-L1 H-Score (per-EMB median, [IQR]) | 5 | (3-16) | 1.5 | (0-3) | 0.034 |

### DISCUSSION

Applying QmIF technology within the cardiac transplant population provides compelling evidence for the feasibility and utility of immune phenotyping for improving the diagnostic and prognostic value of allograft EMB specimens. First, using archived formalin-fixed, paraffin-embedded tissue blocks—some more than 10 years old—successful application of Opal multiplex IF staining technology sufficient for advanced quantitative analysis was achieved in a majority of cases. In designing this experiment, we incorporated a “grade-agnostic” framework using the ‘clinically evident’ and ‘clinically silent’ labels to classify EMBs by the contemporary clinical status of the patient. We hypothesized that QmIF results might suggest mechanisms underlying the frequent discordance between patients' clinical status and the ISHLT grade given to EMB samples. For conventional T-cell markers CD3 and CD8, the QmIF results largely conformed to prior evidence and accepted pathophysiology, and do not significantly facilitate discrimination between patients with and without serious rejection syndromes. High ISHLT grades had markedly higher proportions of CD3+ and CD8+ cells, consistent with the higher overall quantity of basophilic lymphocytes seen in standard H&E slides with high ISHLT histologic grades. Cases grouped a by clinical rejection severity followed a similar pattern, with clinically evident rejection having a higher proportion of these T-cell markers. Since ISHLT grading is primarily predicated on rough quantification of lymphocyte foci (within which CD3+ and CD8+ cells predominate), these findings suggest that misgrading is not the primary reason for clinical-histologic disagreement in our discordant case sub-groups.

The proportion of CD68+ cells was also significantly higher in high ISHLT grade rejection, consistent with several prior reports in renal transplantation which have suggested that macrophages represent a major (and occasionally predominant) cell-line in some cases of cellular rejection^{26, 27}. In contrast, when cases are grouped by clinical status, the proportion of CD68+ cells are significantly lower in EMBs associated with more serious, clinically evident rejection syndromes. This finding is driven almost entirely by the cases with clinical-histologic discordance—clinically silent/high-grade EMBs have by far the highest proportion of CD68+ cells with a prevalence that is ˜40×higher than that seen in clinically evident/low-grade cases (which had by far the lowest proportion). In these clinically silent/high ISHLT grade cases, inspection of the QmIF slide (FIG. 3C4) reveals robust CD68+ staining within the dense cellular infiltrates, which stands in stark contrast to the modest CD68 staining within the cellular infiltrate of the concordant high ISHLT grade case (FIG. 3D4). Since discordant high ISHLT grade cases have comparable (or greater) numbers of CD3+ and CD8+ cells, this visual observation indicates that CD68+ cells are involved in an important ‘late’ protective response, mitigating serious injury to an allograft after lymphocytic infiltration occurs.

The proportion of FoxP3+ cells and cells staining strongly for PD-L1 also differ significantly when cases are grouped by clinical trajectory. Clinically silent rejection events, regardless of ISHLT grade, have significantly higher proportions of both of these anti-inflammatory markers than clinically evident rejection events. These results suggest that the interactions of immune-modulating cell subtypes and pathways play a role in determining whether an initial allo-immune response becomes a clinically serious one. It is worthwhile to highlight that the discordant cases manifest the most extreme proportions of these potentially allograft-protective markers, with the discordant high grade cases containing the highest proportions and the discordant low grade cases containing the lowest. In the case of PD-L1 in particular, this finding appears to be related to the serious myocarditis seen in some cancer patients treated with immune checkpoint inhibitors, as it appears that even low numbers of T-cells (as found in normal myocardium or low ISHLT grades) can induce serious myocardial injury when left unchecked by PD-L1. Visual inspection of QmIF slides further highlights the potential importance of PD-L1, with a diffuse PD-L1+(green) staining pattern seen throughout the myocardium in clinically silent biopsies (FIG. 3; A5 and C5) which is conspicuously absent in clinically evident rejection cases (FIG. 3; B5 and D5). It is also worth highlighting the particularly dense PD-L1 staining seen within the cellular infiltrate of the discordant high ISHLT grade slide (FIG. 3; C5), which may suggest an active and dynamic PD-L1 presence at the site of an acute immune response.

Overall, the QmIF results of EMB cases grouped by clinical trajectory provide an improved approach for accurately assessing the threat to allograft function. Clearly, consideration of more than just a rough count of basophilic immune cells as performed in conventional ISHLT grading is required. This need is reinforced when the concordant low ISHLT grade EMBs are differentiated from one another as ‘future-rejection’ cases vs. ‘never-rejection’ cases, based on whether the patient experiences a serious rejection event in the first three-years post-transplant. The immune profiles of ‘never-rejection’ EMBs are defined by particularly high proportions of PD-L1+ and FoxP3+ cells. In contrast, ‘future-rejection’ EMBs as a whole exhibit lower levels of these allo-protective markers, with striking reductions observed by 3-6 weeks prior to an upcoming clinically-evident rejection. The nearly complete loss of FoxP3+ and PD-L1+ cells in the weeks preceding clinically-evident rejection supports both their utility as biomarkers for identifying patients at high risk for significant rejection events, and the allo-protective role of these markers in heart transplantation.

Mechanistically, the interplay between monocyte lineage cells (CD68+), regulatory T cells (FoxP3+), and PD-L1 expressing cells suggested by our results has been the focus of significant biomedical research, though rarely in the context of organ transplantation⁴⁰⁻⁴⁷. The binding of PD-L1 to PD1 on helper T cells, beyond simple ‘inactivation’ with consequent decreases in inflammatory cytokine production, has been shown to stimulate FoxP3+ expression and effector T cell differentiation into regulatory T cells^{41, 42, 44, 45}. Macrophages and regulatory T cells can interact in a synergistic fashion, with cytokines produced by regulatory T cells encouraging macrophages to differentiate into M2 anti-inflammatory macrophages. In turn, M2 macrophages secrete specific cytokines and express PD-L1, both of which can facilitate further helper T cell differentiation into regulatory T cells. These established mechanisms support the immunologic basis of our findings.

The present research represents provides the means for precision diagnosis and risk stratification in cardiac transplantation. Our results demonstrate the feasibility and translational potential of the QmIF methodology, while identifying several key—and therapeutically targetable—mechanisms involved in determining the severity of undesirable an allo-immune responses.

### Example 2

### Computational Histologic Analysis for the Diagnosis of Heart Transplant Rejection

The results provided in Example 1 suggest that the interaction of immune-modulating cell subtypes and receptors plays a critical role in determining whether an initial allo-immune response becomes a clinically serious one. Put differently, in order to properly assess the threat to overall allograft function, consideration of more than just the raw number of immune cells as seen in traditional H&E slides is required.

Our data demonstrate that the true-negative EMBs (low histologic grade with no evidence of clinical rejection) can be differentiated from one another as ‘future-CAR’ cases vs. ‘never-CAR’ cases, based on whether the patient experiences a serious rejection event within 3-years of transplant. The immune profiles of ‘never-CAR’ patients are defined by particularly high proportions of PD-L1+ and FoxP3+ cells. In contrast, ‘future-CAR’ patients as a whole exhibit lower levels of these allo-protective markers (p<0.01), with a striking decrease when ‘future-CAR’ true-negative EMBs occurring >6 months prior to a serious rejection event are compared to those occurring within 3-6 weeks of serious rejection. The nearly complete loss of FoxP3+ and PD-L1+ cells in the weeks preceding rejection events not only highlights the role of these markers in patient risk-stratification, but also suggests the types of dynamic immuno-biologic processes that are candidates for therapeutic targeting.

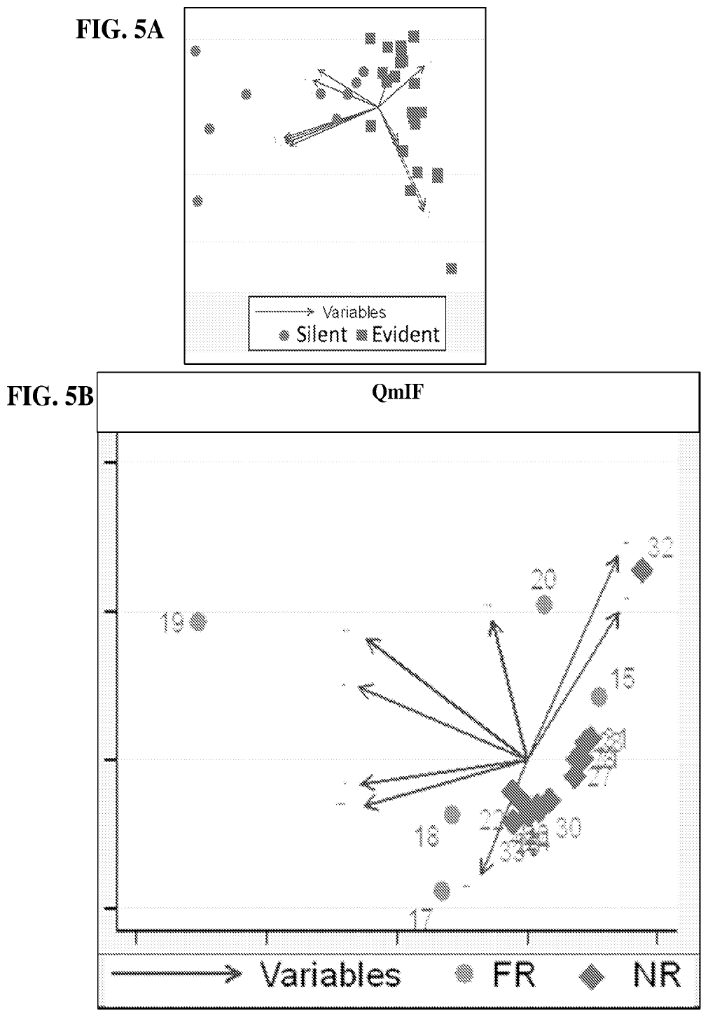

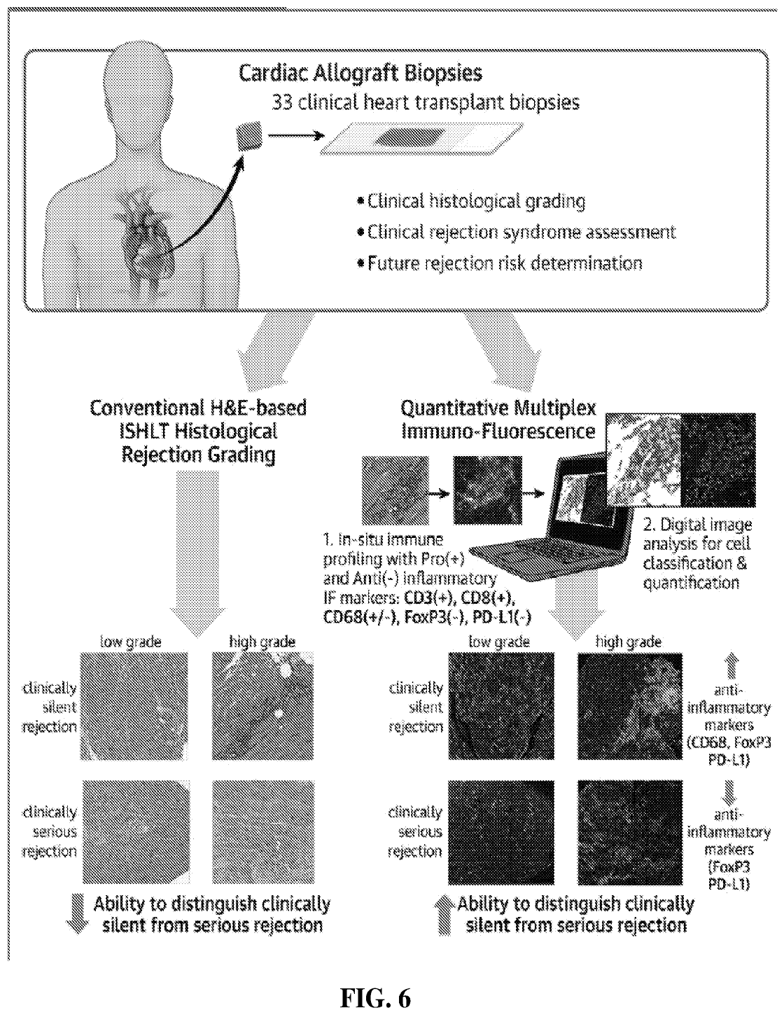

Expanding on the above results, we have performed novel supplementary QmIF image analysis and feature extraction to quantify PD-L1 fluorescence texture, granularity, and pattern/distribution. Combining this novel workflow with custom-built composite statistics generated from the raw cell count data described above, we have conclusively demonstrated the diagnostic and prognostic potential of the QmIF method when paired with appropriate analytical techniques. The biplots in FIGS. 5A and 5B demonstrate the clear segregation on a per-patient/EMB level of applying principal component analysis using the custom-designed composite scores and clusters derived from our statistical and image analysis approaches. FIG. 5A demonstrates diagnostic accuracy, with excellent segregation between clinically silent rejection events and clinically evident rejection events occurring in a manner that is independent of conventional histologic rejection grade. In fact, a single median-linkage cluster analysis utilizing 18 variables segregates clinically evident vs. clinically silent rejection with 100% accuracy. FIG. 5B demonstrates predictive accuracy, with clear segregation of the low-grade, clinically silent EMBs obtained from future-rejector (FR) vs never-rejection (NR) patients. For predicting future rejection, a single score relating PD-L1 to the total population of CD3+ and CD8+ cells accurately segregates 89% (17/19) of cases (‘CompScore2’, with cutoff <1.0 for NR, >1.0 for FR). The results of these subsequent analyses provide strong evidence of the clinical utility of the described approach as a novel diagnostic and prognostic precision-medicine tool.

### Use of Quantitative Multiplex Immunofluorescence (QmIF) on Archived EMB Tissue Blocks to Identify In-Situ Immune-Phenotypes that are Predictive of Future CAR Events

Beyond the significant limitations in CAR diagnostic performance described above, it is important to highlight that neither the established ISHLT histologic grading criteria nor the more recently introduced Omics approaches are designed for predicting future rejection events. Reliable predictions are the basis for prospective risk stratification, which in turn is the basis for establishing personalized approaches to immunosuppression weaning and surveillance EMB schedules. Risk prediction tools have been utilized in clinical medicine for decades to improve the quality of physician decisions when presented with complex, competing-risk scenarios. Within oncology, quantitative morphologic analysis systems and in-situ immune phenotyping have emerged as promising methods for predicting patient outcomes and tailoring therapeutic approaches. In kidney transplant medicine, a clinical EHR prediction algorithm has recently been developed which demonstrated rejection prediction capability in a large validation cohort³⁰. None of these approaches have been utilized to date in cardiac transplant to aid in the prediction of important outcomes. The present example provides compelling evidence from multiple first-in-heart investigations that suggest risk prediction for CAR is achievable.

The International Society for Heart and Lung Transplantation (ISHLT) has recognized the need for “further characterization of the nature of the inflammatory infiltrates” in EMBs, and results from our first-in-heart deployment of QmIF indicate that in-situ immune profiling can identify in-situ processes predictive of future CAR. Accordingly, QmIF will be deployed on the cohort of FR and NR patients previously tested. Quantification of the number, proportion, intensity, and spatial distribution/interactions of different IF markers will be performed. A predictive model consisting of the best quantitative metrics for discriminating between FR and NR can then be developed.

QmIF Methods. QmIF will be performed using the Akoya Biosciences Phenoptics™ commercial platform. This workflow enables multiplex IF of FFPE tissue use of standard, unlabeled primary antibodies via tyramide signal amplification, with subsequent multi-spectral scanning for reliable co-registration of each spectral channel. This method has been used in multiple publications, including our recent first-in-transplant work as described in D⁶⁴⁻⁶⁹. Automated staining and multispectral slide scanning will be performed by Akoya Biosciences with whom we have collaborated on several projects, with digitized slides returned to our team for quantitative image analysis.

TABLE 4
QmIF markers to assess acute and chronic
and pro- and anti-inflammatory mechanisms

| | Pro(+) or | |
|---|---|---|
| | Anti(−) | |
| Marker | Inflammatory | Comment [reference] |
| CD3 | +/− | Pan-T-cell marker, effects depend on subtype⁵ |
| CD8 | + | Cytotoxic T-cell marker, pro-inflammatory^{5, 70-72} |
| FoxP3 | − | Transcription factor of Regulatory T-cell (Treg), allo- |
| | | protective^{5, 73-77} |
| IL17 | + | Marker of pro-inflammatory Th17 cells⁷⁶⁻⁸⁰ |
| PDL1 | − | Ligand for T-cell ‘checkpoint’ PD1, found on multiple |
| | | cell types^{5, 81-87} |
| Gal-9 | +/− | Ligand for ‘checkpoint’ TIM-3, found on multiple cell |
| | | types⁸¹⁻⁹³ |
| C4d | + | Marker of complement activation, associated with |
| | | AMR^{35, 94} |
| CD20 | + | Pan-B-cell marker, presence may suggest chronic |
| | | inflammation^{35, 94} |
| CD68 | +/− | Pan-macrophage marker, variable implications^{5, 35, 94} |
| CD86 | + | Marker of differentiation for proinflammatory M1 |
| | | macrophages^{35, 94-98} |
| D2-40 | +/− | Lymphatic marker, implications vary based on time and |
| | | context⁹⁹⁻¹⁰² |
| CD31 | +/− | Endothelial cell marker, proliferation in chronic |
| | | inflammation^{35, 94} |

OmIF Antibody Panels: Two custom-designed seven-antibody panels will be used to investigate potentially important immunologic cell types and immune effectors. The first panel will focus predominantly on cell-mediated rejection and checkpoint-system markers, and will include CD3, CD8, FoxP3, IL17, PDL1, and Galectin-9 along with a counter-stain. The second panel will focus on chronic and humoral allo-immune markers, and will include CD4, CD20, CD68, CD86, CD31, and D2-40. Together, these panels will enable a comprehensive evaluation of the pro/anti & acute/chronic inflammatory pathways relevant to allograft immune responses in heart tissue, and are informed by strong preliminary data. Table 4 provides further descriptions of the study IF markers, with annotations on marker target, target function, and relevant citations.
Quantitative Image Analysis: QmIF images present novel opportunities and challenges from an image analysis perspective. Comprehensive marker quantitation will be performed as in preliminary work, as well as novel spatial and pattern assessments to fully characterize in-situ interactions of important immune cells and effectors:
Marker Quantitation: The initial step in QmIF workflow is to quantify each individual marker. Mutli-spectral images obtained for this study exist as a compressed stack of single-marker/nuclear counterstain images. This enables easy single-marker cell-count quantitation via color-deconvolution and k-means clustering, similar to the method described above. For PD-L1 and Galectin-9, quantitation will also involve intensity measures for generating intensity quartiles. These quartiles will be used to calculate Histology-Scores (H-Scores), which have been used in many prior studies on immune checkpoints and which were predictive in our published QmIF preliminary research.^{5, 103} For vascular and lymphatic markers CD31 and D2-40, quantitation will also involve measures of the extent of each marker using a hierarchical normalized cuts method pioneered by Dr. Madabhushi's lab for similar tasks in tumor tissue¹⁰⁴.
Marker Co-staining, Co-localization, and Spatial Arrangement: Using the marker-labeled single-cells identified via the above workflow as vertices, graph-based approaches will be employed to provide measures of the spatial relationships between each cell with the same cell (co-staining) and other cells (co-localization) expressing the various IF markers⁴⁸. Because of the variety of scales and patterns these spatial relationships may take, several graphing methods will be employed for this (e.g. Voronoi diagrams, Delaunay drawings).

Texture/Distribution Quantitation: For each marker, the texture and homogeneity of distribution will be assessed via measurements of granularity spectrum^{105, 106} and Haralick features^{107, 108}. From preliminary research, there appears to be significant implications for the pattern of PD-L1 distribution, and we expect that to see additional predictive signals based on the diffuseness vs. focality of marker distributions in this cohort.

### Data Analysis and Statistical Considerations

The image set generated as described above can be split into a training set (n=45 FR patients/n=˜120 slides, n=45 NR patients/n=90 slides) and a held-out validation set (n=15 FR patients/n=˜40 slides and n=15 NR patients/n=30 slides). While the training set of 210 total slides is smaller than that used previously, our preliminary research utilizing QmIF in n=33 slides demonstrated multiple statistically significant univariate predictors of FR vs. NR, and a single composite statistic correlating several cell count/intensity metrics was able to segregate 89% of cases into the appropriate group. Statistically, for the primary analysis, with n=210 QmIF slides in the training set including n=120 (57.1%) associated with FR, this sample size provides the ability to detect an 18% absolute improvement in binary classification over the null hypothesis with 80% power at an alpha of 0.05. Prediction model generation will occur in a similar fashion to the approach described previously, utilizing both extreme gradient boosting and minimum-redundancy/maximum relevancy followed by a mixed-effects model^{58, 61, 62}. Sub-group analyses will be also be performed.

The aforementioned analyses provide a novel CAR risk prediction tool based on a deep characterization of the in-situ immune-biology of allograft tissues. In addition to personalized risk stratification, this model highlights the novel and therapeutically targetable immune processes underlying allograft health and disease.

### Method Details for Example II

Below is a description of additional statistical and image analysis methods used to generate some of the findings described. Additional methods describing the basics of QmIF and the basic primary analyses on cell-count data are described in Example 1.

### Custom Variables:

From raw cells counts and percentages, multiple additional variables were generated. These represented a variety of normalized ratios and inferred cell counts.
PDL1-to-CD8 cell ratio,
CD68-to-CD3 cell ratio
Percent of CD3+ cells that are CD8+
Defined immune cells (sum of CD3+, CD68+, CD8+, FoxP3+ cels),
Non-immune cells (total cells minus defined immune cells),
Percent of Non-immune-cells that are PD-L1+
Percent of Total cell count that are co-stained CD3+/CD8+ cell count

### Novel Quantitative Image Analysis Metrics for PD-L1:

Additional image analysis was performed in CellProfiler 3.1 on raw QmIF slide images corresponding to regions of interest from the primary analysis. These included:
Granularity metrics: This was assessed via measurements of granularity spectrum^{26, 27}, generating 9 novel granularity features for each slide.
Texture/Distribution Metrics: These were assessed via Haralick features^{28, 29}, generating 39 novel texture features related to PD-L1 for each slide (texture angular second moment, texture contrast, texture correlation, texture entropy difference, texture variance difference, texture entropy, texture information measure, texture inverse moment difference, texture entropy, and summary summations of these measures, occurring at three different pixel scales—15 pixels, 75 pixels, and 200 pixels).
Simple Intensity Metrics: Average, median, standard deviation, median absolute deviation, and total (summation of all pixel intensities), and integrated intensity (area covered by PD-L1 stain*total intensity) for PD-L1 staining across all regions of interest for each case.
Co-localization/Nearest Neighbor Metrics: Co-localization was performed via stacked overlay of PD-L1 single marker images with different single marker images from other QmIF markers. Disc dilation at multiple scales was performed to stratify co-localization (5 pixels, 15 pixels, 50 pixels).

### Composite Scores:

Multiple composite scores were generated capturing different relationships between key study markers. These scores have discriminating value alone and in combination with other variables and analytical methods. In particular, CompScore2 alone has excellent discriminating value for assigning FR and NR labels to enable accurate future rejection event prediction.
CompScore 1: [(CD3 cells+CD8 cells)*100]/Total cells
CompScore 2: (CD3 cells+CD8 cells)/(PDL1 cells*(1 PDL1-H-Score))
CompScore 3: (PDL1 cells+H-Score+CD68 cells)/(CD3 cells+CD8+/CD3−cells)
CompScore 4: 10,000*[(FoxP3 cells+H-score+CD68 cells)/(CD3 cells+CD8 cells)]/[(PDL1 cells/total immune cells)+total non-immune cells]
CompScore 5: (FoxP3 cells+PDL1 cells+CD68 cells)/(total immune cells)

### Median-Linkage Cluster Analysis:

Analysis was performed in Stata 15.0 on the following list of 18 variables from the primary analyses and the additional analyses described above:
1. granularity_1pdl1,
2. CompScore1,
3. CompScore2,
4. texture_contrast_pdl1_200_00_avg,
5. cd3 perc_cells_pdl1,
6. granularity_3_pdl1 t
7. exture_contrast_pdl1_15_00_avg,
8. Defined_Immune_Cells,
9. perc_cd3_that_are_foxp3,
10. CompScore3,
11. CompScore4,
12. perc_cd3_that_are_cd8,
13. pdl1_to_cd8_ratio,
14. PDL1_H_score,
15. Integrated_PDL1_intensity_divided_by_total_cells,
16. CompScore5,
17. perc_cd8+cd3+_of_total,
18. cd68_to_cd3_ratio,
   perc_pdl1_of_total_non_immune
   Computational Image Analysis: Digitized pathology slides containing stacks for each fluorescent channel and the brightfield H&E image will be analyzed. For the analysis of the QIF data we will consider several approaches.
   Quantification of Relative Expression of Biomarkers: The HNcut algorithm is an integration of mean shift and normalized cuts within a hierarchical framework developed by our group for accurate and efficient biomarker localization and quantification. The approach can be employed for localizing and quantifying biomarker expression for the individual markers (see Table 5) in the QIF images.
   Spatial Architecture of Biomarkers: Using the location of the biomarkers identified by HNCuts, graph based approaches will be employed for characterization of the relative arrangement of different biomarkers. The HNCut identified locations will form the vertices of a graph. Subsequently for each biomarker, a graph will be constructed with edges connected the spatial location of the biomarker. Depending on the type of edge connection employed, different graph algorithms will then be constructed (e.g. Minimum Spanning Tree, Delaunnay Triangulation, Voronoi). Graph based metrics as described in above will be employed for quantitative description of spatial arrangement of each biomarker.
   Spatial Interplay of Morphologic and QmIF biomarkers: Since the QmIF and H&E images will be co-registered with each other, we will also use the graph based approaches described above to evaluate the interplay between immune cells, myocytes and other cell families on the H&E images with the corresponding biomarkers on the QmIF images. We will employ intersection based graph approaches,⁶⁶ to describe the interplay between morphologic biomarkers and specific molecular phenotypes observed in infiltrating immune cells (T-cells, B-cells, macrophages) and endothelial cells (analogous to the application in prostate cancer in previous studies).

### Image Analysis Workflow:

We have developed a framework for extracting an array of morphologic features which describe rejection histology and intend to expand our analysis to include additional targets and workflows. Histologic CAR grading has codified criteria upon which to base the feature extraction approach (eg. quantitation of lymphocyte foci and their spatial relationship to myocytes). There is no such pre-defined ruleset for predicting future CAR, so in addition to re-purposing components of the existing CACHE-Grader pipeline, a broader approach to feature extraction has been developed. This approach will identify novel morphologic elements that were not relevant or necessary for reproducing ISHLT grades. Specifically, we will perform a broader characterization of myocyte size, shape and orientation, myocyte nuclei size, shape, and texture, interstitial/stromal metrics, and characterization of micro-vasculature via the following methods:

Tissue Compartment Segmentation into Myocytes, Lymphocytes, Stroma, and Vasculature.

After applying color deconvolution algorithm⁴⁰ to separate different stain-pigments, K-means clustering⁴¹ can be used to assign three classes (myocytes, interstitium, and non-myocyte nuclei) to each pixel as described in our previous work. Following segmentation into these three classes, the interstitial compartment will undergo further segmentation to identify small vessels using a neural network approach⁴². This sequential approach is favored based on prior efforts in which unstained areas around myocyte nuclei have impacted vascular segmentation.

Isolating lymphocyte clusters. Since biologically relevant lymphocyte populations typically exist as colonies rather than lone cells, and because single nuclei in isolation can represent several cell types (some leukocytes, some non-leukocytes), identification and isolation of lymphocyte clusters is a necessary process. We have achieved this as we have in prior work by performing disc-dilation, area thresholding, and proximity graphs. Variables based on identifying these clusters were the strongest predictors of rejection grade in our early analyses.
Myocyte and myocyte nuclei shape, pattern, and density metrics: Morphologic patterns derived from the shape⁴³⁻⁴⁷ orientation^{44, 48-53}, and density^{48, 54-57} of cells and their nuclei can be extracted from H&E slides using established methods. Quantitative variables based on these patterns have prognostic and predictive value in oncology, and should be informative on the health and fate of allograft myocardium.
Identifying Foci Neighborhoods. The local neighborhood of a lymphocyte informs histologic grading, with lymphocytes in the endocardium vs. constrained to the interstitium vs. encroaching upon myocyte borders being explicitly described in formal ISHLT grading criteria. Spatial analysis of the lymphocyte location will be based on compartment segmentation, and interactions with myocytes can be assessed via proximity graphs.
Sample Size, Data Analysis and Statistical Considerations: For predictive model development, the image set can be split into a training set (n=169 FR patients/n=−460 slides, n=336 NR patients/n=672 slides) and a held-out validation set (n=56 FR patients/n=−140 slides and n=114 NR patients/n=228 slides).

The primary model can incorporate all EMBs, and is designed to predict the likelihood of the FR vs. NR class. Predictive model generation can be conducted in both an unsupervised fashion and a supervised fashion, with the approaches compared to select the best final model. For unsupervised model generation, extreme gradient boosting⁵⁸ will be employed on the entire H&E morphologic feature set generated via the image analysis workflow. Extreme gradient boosting is designed to handle large array data, and performed well in feasibility testing on 154 quantitative features generated by the CACHE-Grader pipeline (refer to D.4). For added granularity, we will also employ a minimum redundancy maximum relevance method^{59, 60} followed by forward-selection within a multivariate mixed effects model^{61, 62} to generate a parsimonious and fully transparent prediction model. It should be noted that a mixed effects model has been chosen to take into account potential temporal and spatial correlations resulting from repeated measures of the same individual patient (eg. more than 1 slide per patient). The predictive performance of the supervised and unsupervised models will be compared in the held-out set.

Important sub-analyses will also be performed. Specifically, EMB slides will be sub-analyzed by acquisition date relative to the date of transplant and, for FR patients, the date of incident CAR. Modeling of quantitative morphologic features extracted from EMBs obtained 1-month and 3-months after transplant for FR and NR patients will be performed to assess whether CAR risk assessments (and thus, post-transplant management decisions) can be made during the early post-transplant period. Additionally, for the FR group, modeling of morphologic features from the EMB events in the weeks preceding incident CAR will be performed, and compared with EMBs from earlier time points to understand dynamic elements suggestive of impending CAR.

The aforementioned analyses provide a novel CAR risk prediction tool based on a deep characterization of the in-situ immune-biology of allograft tissues. In addition to personalized risk stratification, this model highlights the novel and therapeutically targetable immune processes underlying allograft health and disease.

### REFERENCES FOR EXAMPLE 1

1. Klingenberg, R., et al., Allograft rejection of ISHLT grade >/=3A occurring late after heart transplantation—a distinct entity? J Heart Lung Transplant, 2003. 22 (9): p. 1005-13.
2. Kobashigawa, J. A., The Search for a Gold Standard to Detect Rejection in Heart Transplant Patients: Are We There Yet? Circulation, 2017. 135 (10): p. 936-938.
3. Pham, M. X., et al., Gene-expression profiling for rejection surveillance after cardiac transplantation. N Engl J Med, 2010. 362 (20): p. 1890-900.
4. Dandel, M., et al., Left ventricular dysfunction during cardiac allograft rejection: early diagnosis, relationship to the histological severity grade, and therapeutic implications. Transplant Proc, 2002. 34 (6): p. 2169-73.
5. Stewart, S., et al., Revision of the 1990 working formulation for the standardization of nomenclature in the diagnosis of heart rejection. J Heart Lung Transplant, 2005. 24 (11): p. 1710-20.
6. Wang, C., et al., Immune profiling of microsatellite instability-high and polymerase ε (POLE)-mutated metastatic colorectal tumors identifies predictors of response to anti-PD-1 therapy. Journal of Gastrointestinal Oncology, 2018. 9 (3): p. 404-415.
7. van den Berg, T. K. and T. Valerius, Myeloid immune-checkpoint inhibition enters the clinical stage. Nature Reviews Clinical Oncology, 2018.
8. Galon, J. and D. Bruni, Approaches to treat immune hot, altered and cold tumours with combination immunotherapies. Nature Reviews Drug Discovery, 2019.
9. Wang, Y. L., J. M. Gao, and L. Z. Xing, Therapeutic potential of Oroxylin A in rheumatoid arthritis. Int Immunopharmacol, 2016. 40: p. 294-299.
10. Wehr, P., et al., Dendritic cells, T cells and their interaction in Rheumatoid arthritis. Clin Exp Immunol, 2018.
11. Kim, N. and H. S. Kim, Targeting Checkpoint Receptors and Molecules for Therapeutic Modulation of Natural Killer Cells. Front Immunol, 2018. 9: p. 2041.
12. Burugu, S., A. R. Dancsok, and T. O. Nielsen, Emerging targets in cancer immunotherapy. Semin Cancer Biol, 2018. 52 (Pt 2): p. 39-52.
13. Stack, E. C., et al., Multiplexed immunohistochemistry, imaging, and quantitation: A review, with an assessment of Tyramide signal amplification, multispectral imaging and multiplex analysis. Methods, 2014. 70 (1): p. 46-58.
14. Parra, E. R., et al., Validation of multiplex immunofluorescence panels using multispectral microscopy for immune-profiling of formalin-fixed and paraffin-embedded human tumor tissues. Scientific Reports, 2017. 7 (1): p. 13380.
15. Blom, S., et al., Systems pathology by multiplexed immunohistochemistry and whole-slide digital image analysis. Scientific reports, 2017. 7 (1): p. 15580-15580.
16. Mansfield, J., et al., Validation of multiplex immunofluorescence for use in analysis of tumour infiltrating lymphocytes. Vol. 3. 2015. p⁴¹1.
17. Gorris, M. A. J., et al., Eight-Color Multiplex Immunohistochemistry for Simultaneous Detection of Multiple Immune Checkpoint Molecules within the Tumor Microenvironment. J Immunol, 2018. 200 (1): p. 347-354.
18. Harper, S. J. F., et al., CD8 T-cell recognition of acquired alloantigen promotes acute allograft rejection. Proceedings of the National Academy of Sciences of the United States of America, 2015. 112 (41): p. 12788-12793.
19. Rocha, P. N., et al., Effector mechanisms in transplant rejection. Immunol Rev, 2003. 196: p. 51-64.
20. Le Moine, A., M. Goldman, and D. Abramowicz, Multiple pathways to allograft rejection. Transplantation, 2002. 73 (9): p. 1373-81.
21. Bestard, O., et al., Presence of FoxP3+ regulatory T Cells predicts outcome of subclinical rejection of renal allografts. J Am Soc Nephrol, 2008. 19 (10): p. 2020-6.
22. Chung, B. H., et al., Clinical significance of the ratio between FOXP3 positive regulatory T cell and interleukin-17 secreting cell in renal allograft biopsies with acute T-cell-mediated rejection. Immunology, 2012. 136 (3): p. 344-51.
23. Hua, J., et al., Pathological conversion of regulatory T cells is associated with loss of allotolerance. Sci Rep, 2018. 8 (1): p. 7059.
24. Raimondi, G., et al., Mammalian target of rapamycin inhibition and alloantigen-specific regulatory T cells synergize to promote long-term graft survival in immunocompetent recipients. J Immunol, 2010. 184 (2): p. 624-36.
25. Liao, T., et al., In Vivo Attenuation of Antibody-Mediated Acute Renal Allograft Rejection by Ex Vivo TGF-β-Induced CD4(+)Foxp3 (+) Regulatory T Cells. Frontiers in immunology, 2017. 8: p. 1334-1334.
26. Tinckam, K. J., O. Djurdjev, and A. B. Magil, Glomerular monocytes predict worse outcomes after acute renal allograft rejection independent of C4d status. Kidney Int, 2005. 68 (4): p. 1866-74.
27. Girlanda, R., et al., Monocyte infiltration and kidney allograft dysfunction during acute rejection. Am J Transplant, 2008. 8 (3): p. 600-7.
28. Alhamad, T., et al., Checkpoint Inhibitors in Kidney Transplant Recipients and the Potential Risk of Rejection. Am J Transplant, 2016. 16 (4): p. 1332-3.
29. Goldman, J. W., et al., PD1 checkpoint inhibition in solid organ transplants: 2 sides of a coin—case report. BMC Nephrology, 2018. 19 (1): p. 210.
30. Lipson, E. J., et al., Tumor Regression and Allograft Rejection after Administration of Anti-PD-1. N Engl J Med, 2016. 374 (9): p. 896-8.
31. Pilat, N., M. H. Sayegh, and T. Wekerle, Costimulatory pathways in transplantation. Seminars in Immunology, 2011. 23 (4): p. 293-303.
32. Tanaka, K., et al., PDL1 is required for peripheral transplantation tolerance and protection from chronic allograft rejection. Journal of immunology (Baltimore, Md.: 1950), 2007. 179 (8): p. 5204-5210.
33. Schutte-Nutgen, K., et al., Divergent Function of Programmed Death-Ligand 1 in Donor Tissue versus Recipient Immune System in a Murine Model of Bronchiolitis Obliterans. The American Journal of Pathology, 2017. 187 (6): p. 1368-1379.
34. Costanzo, M. R., et al., The International Society of Heart and Lung Transplantation Guidelines for the care of heart transplant recipients. J Heart Lung Transplant, 2010. 29 (8): p. 914-56.
35. Kobashigawa, J. A., et al., Tacrolimus with mycophenolate mofetil (MMF) or sirolimus vs. cyclosporine with MMF in cardiac transplant patients: 1-year report. Am J Transplant, 2006. 6 (6): p. 1377-86.
36. Eisen, H. J., et al., Everolimus for the prevention of allograft rejection and vasculopathy in cardiac-transplant recipients. N Engl J Med, 2003. 349 (9): p. 847-58.
37. Kobashigawa, J., et al., Randomized pilot trial of gene expression profiling versus heart biopsy in the first year after heart transplant: early invasive monitoring attenuation through gene expression trial. Circ Heart Fail, 2015. 8 (3): p. 557-64.
38. Igarashi, T., et al., Scoring of PD-L1 expression intensity on pulmonary adenocarcinomas and the correlations with clinicopathological factors. ESMO Open, 2016. 1 (4): p. e000083.
39. Berry, G. J., et al., The 2013 International Society for Heart and Lung Transplantation Working Formulation for the standardization of nomenclature in the pathologic diagnosis of antibody-mediated rejection in heart transplantation. J Heart Lung Transplant, 2013. 32 (12): p. 1147-62.
40. Schmidt, A., et al., Human macrophages induce CD4(+)Foxp3(+) regulatory T cells via binding and re-release of TGF-beta. Immunol Cell Biol, 2016. 94 (8): p. 747-62.
41. Amarnath, S., et al., The PDL1-PD1 axis converts human TH1 cells into regulatory T cells. Sci Transl Med, 2011. 3 (111): p. 11lral20.
42. Francisco, L. M., et al., PD-L1 regulates the development, maintenance, and function of induced regulatory T cells. J Exp Med, 2009. 206 (13): p. 3015-29.
43. Romano, M., et al., Expanded Regulatory T Cells Induce Alternatively Activated Monocytes With a Reduced Capacity to Expand T Helper-17 Cells. Frontiers in Immunology, 2018. 9 (1625).
44. Gianchecchi, E. and A. Fierabracci, Inhibitory Receptors and Pathways of Lymphocytes: The Role of PD-1 in Treg Development and Their Involvement in Autoimmunity Onset and Cancer Progression. Frontiers in immunology, 2018. 9: p. 2374-2374.
45. Miao, X., et al., PD-L1 reverses depigmentation in Pmel-1 vitiligo mice by increasing the abundance of Tregs in the skin. Sci Rep, 2018. 8 (1): p. 1605.
46. Zhao, Y., et al., Macrophage subpopulations and their impact on chronic allograft rejection versus graft acceptance in a mouse heart transplant model. American journal of transplantation: official journal of the American Society of Transplantation and the American Society of Transplant Surgeons, 2018. 18 (3): p. 604-616.
47. Grabie, N., A. H. Lichtman, and R. Padera, T cell checkpoint regulators in the heart. Cardiovascular Research, 2019. 115 (5): p. 869-877.
48. Nirschl, J. J., et al., A deep-learning classifier identifies patients with clinical heart failure using whole-slide images of H&E tissue. PLoS One, 2018. 13 (4): p. e0192726.
49. Peyster, E. G., A. Madabhushi, and K. B. Margulies, Advanced Morphologic Analysis for Diagnosing Allograft Rejection: The Case of Cardiac Transplant Rejection. Transplantation, 2018. 102 (8): p. 1230-1239.
50. Hutloff, A., et al., ICOS is an inducible T-cell co-stimulator structurally and functionally related to CD28. Nature, 1999. 397 (6716): p. 263-266.

### REFERENCES FOR EXAMPLE 2

1. Lund, L. H., et al., The registry of the International Society for Heart and Lung Transplantation: thirty-first official adult heart transplant report—2014; focus theme: retransplantation. J Heart Lung Transplant, 2014. 33 (10): p. 996-1008.
2. Costanzo, M. R., et al., The International Society of Heart and Lung Transplantation Guidelines for the care of heart transplant recipients. J Heart Lung Transplant, 2010. 29 (8): p. 914-56.
3. Billingham, M. E., et al., A working formulation for the standardization of nomenclature in the diagnosis of heart and lung rejection: Heart Rejection Study Group. The International Societyfor Heart Transplantation. J Heart Transplant, 1990. 9 (6): p. 587-93.
4. Angelini, A., et al., A web-based pilot study of inter-pathologist reproducibility using the ISHLT 2004 working formulation for biopsy diagnosis of cardiac allograft rejection: the European experience. J Heart Lung Transplant, 2011. 30 (11): p. 1214-20.
5. Crespo-Leiro, M. G., et al., Concordance among pathologists in the second Cardiac Allograft Rejection Gene Expression Observational Study (CARGO II). Transplantation, 2012. 94 (11): p. 1172-7.
6. Klingenberg, R., et al., Allograft rejection of ISHLT grade >/=3A occurring late after heart transplantation—a distinct entity? J Heart Lung Transplant, 2003. 22 (9): p. 1005-13.
7. Kobashigawa, J. A., The Search for a Gold Standard to Detect Rejection in Heart Transplant Patients: Are We There Yet? Circulation, 2017. 135 (10): p. 936-938.
8. Pham, M. X., et al., Gene-expression profiling for rejection surveillance after cardiac transplantation. N Engl J Med, 2010. 362 (20): p. 1890-900.
9. Dandel, M., et al., Left ventricular dysfunction during cardiac allograft rejection: early diagnosis, relationship to the histological severity grade, and therapeutic implications. Transplant Proc, 2002. 34 (6): p. 2169-73.
10. Stewart, S., et al., Revision of the 1990 working formulation for the standardization of nomenclature in the diagnosis of heart rejection. J Heart Lung Transplant, 2005. 24 (11): p. 1710-20.
11. Stack, E. C., et al., Multiplexed immunohistochemistry, imaging, and quantitation: A review, with an assessment of Tyramide signal amplification, multispectral imaging and multiplex analysis. Methods, 2014. 70 (1): p. 46-58.
12. Parra, E. R., et al., Validation of multiplex immunofluorescence panels using multispectral microscopy for immune-profiling of formalin-fixed and paraffin-embedded human tumor tissues. Scientific Reports, 2017. 7 (1): p. 13380.
13. Blom, S., et al., Systems pathology by multiplexed immunohistochemistry and whole-slide digital image analysis. Scientific reports, 2017. 7 (1): p. 15580-15580.
14. Mansfield, J., et al., Validation of multiplex immunofluorescence for use in analysis of tumour infiltrating lymphocytes. Vol. 3. 2015. p 411.
15. Gorris, M. A. J., et al., Eight-Color Multiplex Immunohistochemistry for Simultaneous Detection of Multiple Immune Checkpoint Molecules within the Tumor Microenvironment. J Immunol, 2018. 200 (1): p. 347-354.
16. Lesinski, G. B., et al., Interleukin-6/STAT3 Signaling is Prominent and Associated with Reduced Overall Survival in p 16 Negative Oropharyngeal Squamous Cell Carcinoma. Head Neck Pathol, 2018.
17. Savage, N. D. L., et al., Human Anti-Inflammatory Macrophages Induce Foxp3&lt;sup&gt; +&lt;/sup&gt;GITR&lt;sup&gt; +&lt;/sup&gt; CD25&lt;sup&gt; +&lt;/sup& gt; Regulatory T Cells, Which Suppress via Membrane-Bound TGFβ-1. The Journal of Immunology, 2008. 181 (3): p. 2220.
18. Schmidt, A., et al., Human macrophages induce CD4(+)Foxp3(+) regulatory T cells via binding and re-release of TGF-beta. Immunol Cell Biol, 2016. 94 (8): p. 747-62.
19. Amarnath, S., et al., The PDL1-PD1 axis converts human TH1 cells into regulatory T cells. Sci Transl Med, 2011. 3 (111): p. 11lral20.
20. Francisco, L. M., et al., PD-L1 regulates the development, maintenance, and function of induced regulatory T cells. J Exp Med, 2009. 206 (13): p. 3015-29.
21. Romano, M., et al., Expanded Regulatory T Cells Induce Alternatively Activated Monocytes With a Reduced Capacity to Expand T Helper-17 Cells. Frontiers in Immunology, 2018. 9 (1625).
22. Gianchecchi, E. and A. Fierabracci, Inhibitory Receptors and Pathways of Lymphocytes: The Role of PD-1 in Treg Development and Their Involvement in Autoimmunity Onset and Cancer Progression. Frontiers in immunology, 2018. 9: p. 2374-2374.
23. Miao, X., et al., PD-L1 reverses depigmentation in Pmel-1 vitiligo mice by increasing the abundance of Tregs in the skin. Sci Rep, 2018. 8 (1): p. 1605.
24. Zhao, Y., et al., Macrophage subpopulations and their impact on chronic allograft rejection versus graft acceptance in a mouse heart transplant model. American journal of transplantation: official journal of the American Society of Transplantation and the American Society of Transplant Surgeons, 2018. 18 (3): p. 604-616.
25. Grabie, N., A. H. Lichtman, and R. Padera, T cell checkpoint regulators in the heart. Cardiovascular Research, 2019. 115 (5): p. 869-877.
26. Maragos, P., Pattern spectrum and multiscale shape representation. IEEE Trans Pattern Anal Mach Intell. Pattern Analysis and Machine Intelligence, IEEE Transactions on, 1989. 11: p. 701-716.
27. Vincent, L., “Granulometries and Opening Trees”. Fundamenta Informaticae, 2000. 41 (1-2): p. 57-90.
28. Haralick, R. M., Statistical and structural approaches to texture. Proceedings of the IEEE, 1979. 67 (5): p. 786-804.
29. Haralick, R. M., K. Shanmugam, and I. Dinstein, Textural Features for Image Classification. IEEE Transactions on Systems, Man, and Cybernetics, 1973. SMC-3 (6): p. 610-621.

### REFERENCES FOR EXAMPLE 2

1. Arabyarmohammadi S, J. A., Peyster EG, Azarianpour S, Margulies KB, Madabhushi A., Assessment of Cardiac Rejection Grade Via Machine Learning Derived Features from Digital Biopsy Images, in American Heart Association Scientific Sessions 2019. 2019: Philadelphia, PA.
2. Corbin, E. A., et al., Tunable and Reversible Substrate Stiffness Reveals a Dynamic Mechanosensitivity of Cardiomyocytes. ACS Appl Mater Interfaces, 2019. 11 (23): p. 20603-20614.
3. Nirschl, J. J., et al., A deep-learning classifier identifies patients with clinical heart failure using whole-slide images of H&E tissue. PLOS ONE, 2018. 13 (4): p. e0192726.
4. Peyster E, A. S., Janowczyk A, Sekulic M, Cassol C, Feldman M, Margulies K, Madabhushi A., Computer-Assisted Cardiac Histologic Evaluation (CACHE) for Grading Cardiac Allograft Rejection Am J Transplant. 20 ((suppl 3)).
5. Peyster, E. G., et al., In Situ Immune Profiling of Heart Transplant Biopsies Improves Diagnostic Accuracy and Rejection Risk Stratification. JACC: Basic to Translational Science, 2020. 5 (4): p. 328-340.
6. Lund, L. H., et al., The registry of the International Society for Heart and Lung Transplantation: thirty-first official adult heart transplant report—2014; focus theme: retransplantation. J Heart Lung Transplant, 2014. 33 (10): p. 996-1008.
7. Costanzo, M. R., et al., The International Society of Heart and Lung Transplantation Guidelines for the care of heart transplant recipients. J Heart Lung Transplant, 2010. 29 (8): p. 914-56.
8. Billingham, M. E., et al., A working formulation for the standardization of nomenclature in the diagnosis of heart and lung rejection: Heart Rejection Study Group. The International Society for Heart Transplantation. J Heart Transplant, 1990. 9 (6): p. 587-93.
9. Strecker, T., et al., Endomyocardial biopsy for monitoring heart transplant patients: 11-years-experience at a german heart center. International journal of clinical and experimental pathology, 2012. 6 (1): p. 55-65.
10. Saraiva, F., et al., Complications of endomyocardial biopsy in heart transplant patients: a retrospective study of 2117 consecutive procedures. Transplant Proc, 2011. 43 (5): p. 1908-12.
11. Rodriguez, E. R., The pathology of heart transplant biopsy specimens: revisiting the 1990 ISHLT working formulation. J Heart Lung Transplant, 2003. 22 (1): p. 3-15.
12. Winters, G. L., The challenge of endomyocardial biopsy interpretation in assessing cardiac allograft rejection. Curr Opin Cardiol, 1997. 12 (2): p. 146-52.
13. Winters, G. L., C. C. Marboe, and M. E. Billingham, The International Society for Heart and Lung Transplantation grading system for heart transplant biopsy specimens: clarification and commentary. J Heart Lung Transplant, 1998. 17 (8): p. 754-60.
14. Stewart, S., et al., Revision of the 1990 working formulation for the standardization of nomenclature in the diagnosis of heart rejection. J Heart Lung Transplant, 2005. 24 (11): p. 1710-20.
15. Crespo-Leiro, M. G., et al., Concordance among pathologists in the second Cardiac Allograft Rejection Gene Expression Observational Study (CARGO II). Transplantation, 2012. 94 (11): p. 1172-7.
16. Marboe, C. C., et al., Nodular endocardial infiltrates (Quilty lesions) cause significant variability in diagnosis of ISHLT Grade 2 and 3A rejection in cardiac allograft recipients. J Heart Lung Transplant, 2005. 24 (7 Suppl): p. S219-26.
17. Fishbein, M. C. and J. Kobashigawa, Biopsy-negative cardiac transplant rejection: etiology, diagnosis, and therapy. Curr Opin Cardiol, 2004. 19 (2): p. 166-9.
18. Angelini, A., et al., A web-based pilot study of inter-pathologist reproducibility using the ISHLT 2004 working formulation for biopsy diagnosis of cardiac allograft rejection: the European experience. J Heart Lung Transplant, 2011. 30 (11): p. 1214-20.
19. Klingenberg, R., et al., Allograft rejection of ISHLT grade >/=3A occurring late after heart transplantation—a distinct entity? J Heart Lung Transplant, 2003. 22 (9): p. 1005-13.
20. Kobashigawa, J. A., The Search for a Gold Standard to Detect Rejection in Heart Transplant Patients: Are We There Yet? Circulation, 2017. 135 (10): p. 936-938.
21. Pham, M. X., et al., Gene-expression profiling for rejection surveillance after cardiac transplantation. N Engl J Med, 2010. 362 (20): p. 1890-900.
22. Dandel, M., et al., Left ventricular dysfunction during cardiac allograft rejection: early diagnosis, relationship to the histological severity grade, and therapeutic implications. Transplant Proc, 2002. 34 (6): p. 2169-73.
23. Butler, C. R., et al., Cardiovascular magnetic resonance in the diagnosis of acute heart transplant rejection: a review. J Cardiovasc Magn Reson, 2009. 11: p. 7.
24. Butler, C. R., et al., Correlation of cardiovascular magnetic resonance imaging findings and endomyocardial biopsy results in patients undergoing screening for heart transplant rejection. J Heart Lung Transplant, 2015. 34 (5): p. 643-50.
25. Wu, Y. L., et al., Noninvasive evaluation of cardiac allograft rejection by cellular and functional cardiac magnetic resonance. JACC Cardiovasc Imaging, 2009. 2 (6): p. 731-41.
26. Deng, M. C., et al., Noninvasive discrimination of rejection in cardiac allograft recipients using gene expression profiling. Am J Transplant, 2006. 6 (1): p. 150-60.
27. Crespo-Leiro, M. G., et al., Clinical usefulness of gene-expression profile to rule out acute rejection after heart transplantation: CARGO II. Eur Heart J, 2016. 37 (33): p. 2591-601.
28. Halloran, P. F., et al., Building a tissue-based molecular diagnostic system in heart transplant rejection: The heart Molecular Microscope Diagnostic (MMDx) System. J Heart Lung Transplant, 2017. 36 (11): p. 1192-1200.
29. Ling, X., et al., Can immune cell function assay identify patients at risk of infection or rejection? A meta-analysis. Transplantation, 2012. 93 (7): p. 737-43.
30. Loupy, A., et al., Prediction systemfor risk of allograft loss in patients receiving kidney transplants: international derivation and validation study. BMJ, 2019. 366: p. 14923.
31. Nirschl, J., et al. Deep Learning Classifier to Predict Cardiac Failure from Whole-Slide H&E Images. in LABORATORY INVESTIGATION. 2017. NATURE PUBLISHING GROUP 75 VARICK ST, 9TH FLR, NEW YORK, NY 10013-1917 USA.
32. Peyster, E. G., A. Madabhushi, and K. B. Margulies, Advanced Morphologic Analysis for Diagnosing Allograft Rejection: The Case of Cardiac Transplant Rejection. Transplantation, 2018. 102 (8): p. 1230-1239.
33. Duong Van Huyen, J. P., et al., The XVth Banff Conference on Allograft Pathology The Banff Workshop Heart Report: Improving the Diagnostic Yield from Endomyocardial Biopsies and Quilty Effect Revisited. Am J Transplant, 2020.
34. Jeffrey J. Nirschl, A. J., Eliot G. Peyster, Renee Frank, Kenneth B. Margulies, Michael D. Feldman, Anant Madabhushi, Deep Learning Tissue Segmentation in Cardiac Histopathology Images, in Deep Learning for Medical Image Analysis, H. G. S. Kevin Zhou, Dinggang Shen, Editor. 2017. p. pp. 179-195.
35. Berry, G. J., et al., The 2013 International Society for Heart and Lung Transplantation Working Formulation for the standardization of nomenclature in the pathologic diagnosis of antibody-mediated rejection in heart transplantation. J Heart Lung Transplant, 2013. 32 (12): p. 1147-62.
36. Janowczyk, A., A. Basavanhally, and A. Madabhushi, Stain Normalization using Sparse AutoEncoders (StaNoSA): Application to digital pathology. Computerized medical imaging and graphics: the official journal of the Computerized Medical Imaging Society, 2017. 57: p. 50-61.
37. Janowczyk, A. and A. Madabhushi, Deep learning for digitalpathology image analysis: A comprehensive tutorial with selected use cases. Journal of Pathology Informatics, 2016. 7 (1): p. 29-29.
38. Anghel, A., et al., A High-Performance System for Robust Stain Normalization of Whole-Slide Images in Histopathology. Frontiers in medicine, 2019. 6: p. 193-193.
39. Janowczyk, A., et al., HistoQC: An Open-Source Quality Control Tool for Digital Pathology Slides. JCO Clinical Cancer Informatics, 2019 (3): p. 1-7.
40. Ruifrok, A. and D. Johnston, Ruifrok A C, Johnston D A. Quantification of histochemical staining by color deconvolution. Anal Quant Cytol Histol 23: 291-299. Analytical and quantitative cytology and histology/the International Academy of Cytology [and] American Society of Cytology, 2001. 23: p. 291-9.
41. MacQueen, J. B. Some Methods for Classification and Analysis of MultiVariate Observations. in Proc. of the fifth Berkeley Symposium on Mathematical Statistics and Probability. 1967. University of California Press.
42. Yi, F., et al., Microvessel prediction in H&E Stained Pathology Images using fully convolutional neural networks. BMC bioinformatics, 2018. 19 (1): p. 64-64.
43. Antunes, J., et al., Coregistration of Preoperative MRI with Ex Vivo Mesorectal Pathology Specimens to Spatially Map Post-treatment Changes in Rectal Cancer Onto In Vivo Imaging: Preliminary Findings. Acad Radiol, 2018. 25 (7): p. 833-841.
44. Lu, C., et al., An oral cavity squamous cell carcinoma quantitative histomorphometric-based image classifier of nuclear morphology can risk stratify patients for disease-specific survival. Mod Pathol, 2017. 30 (12): p. 1655-1665.
45. Wang, X., et al., Prediction of recurrence in early stage non-small cell lung cancer using computer extracted nuclear features from digital H&E images. Sci Rep, 2017. 7 (1): p. 13543.
46. Carleton, N. M., et al., Advances in the computational and molecular understanding of the prostate cancer cell nucleus. J Cell Biochem, 2018. 119 (9): p. 7127-7142.
47. Lu, C., et al., Nuclear shape and orientation features from H&E images predict survival in early-stage estrogen receptor-positive breast cancers. Lab Invest, 2018. 98 (11): p. 1438-1448.
48. Corredor, G., et al., Spatial architecture and arrangement of tumor-infiltrating lymphocytes for predicting likelihood of recurrence in early-stage non-small cell lung cancer. Clinical Cancer Research, 2018: p. clincanres.2013.2018.
49. Doyle, S., et al., Cascaded discrimination of normal, abnormal, and confounder classes in histopathology: Gleason grading of prostate cancer. BMC Bioinformatics, 2012. 13: p. 282.
50. Golugula, A., et al., Supervised regularized canonical correlation analysis: integrating histologic and proteomic measurements for predicting biochemical recurrence following prostate surgery. BMC Bioinformatics, 2011. 12: p. 483.
51. Lee, G., et al., Co-occurring gland angularity in localized subgraphs: predicting biochemical recurrence in intermediate-risk prostate cancer patients. PloS one, 2014. 9 (5): p. e97954-e97954.
52. Leo, P., et al. Combination of nuclear NF-kB/p65 localization and gland morphological features from surgical specimens is predictive of early biochemical recurrence in prostate cancer patients. in SPIE Medical Imaging. 2018. SPIE.
53. Lu, C., et al., Feature Driven Local Cell Graph (FeDeG): Predicting Overall Survival in Early Stage Lung Cancer. 2018. p. 407-416.
54. Gong, C., et al., Quantitative Characterization of CD8+ T Cell Clustering and Spatial Heterogeneity in Solid Tumors. Frontiers in Oncology, 2019. 8 (649).
55. Corredor, G., et al., Spatial Architecture and Arrangement of Tumor-Infiltrating Lymphocytes for Predicting Likelihood of Recurrence in Early-Stage Non-Small Cell Lung Cancer. Clinical cancer research: an official journal of the American Association for Cancer Research, 2019. 25 (5): p. 1526-1534.
56. Saltz, J., et al., Spatial Organization and Molecular Correlation of Tumor-Infiltrating Lymphocytes Using Deep Learning on Pathology Images. Cell Rep, 2018. 23 (1): p. 181-193.e7.
57. Yuan, Y., Modelling the spatial heterogeneity and molecular correlates of lymphocytic infiltration in triple-negative breast cancer. J R Soc Interface, 2015. 12 (103).
58. Chen, T. and C. Guestrin, XGBoost: A Scalable Tree Boosting System. 2016. 785-794.
59. Ding, C. and H. Peng, MINIMUM REDUNDANCY FEATURE SELECTION FROM MICROARRAY GENE EXPRESSION DATA. Journal of Bioinformatics and Computational Biology, 2005. 03(02): p. 185-205.
60. Mandal, M. and A. Mukhopadhyay, An Improved Minimum Redundancy Maximum Relevance Approach for Feature Selection in Gene Expression Data. Procedia Technology, 2013. 10: p. 20-27.
61. Fieuws, S. and G. Verbeke, Joint modelling of multivariate longitudinal profiles: pitfalls of the random-effects approach. Statistics in Medicine, 2004. 23 (20): p. 3093-3104.
62. Thiebaut, R., et al., Bivariate linear mixed models using SAS proc MIXED. Computer Methods and Programs in Biomedicine, 2002. 69 (3): p. 249-256.
63. Sammut, C. W. G. I., Encylopedia of Mechine Learning. 1st ed ed. 2011: Springer p.
64. Stack, E. C., et al., Multiplexed immunohistochemistry, imaging, and quantitation: A review, with an assessment of Tyramide signal amplification, multispectral imaging and multiplex analysis. Methods, 2014. 70 (1): p. 46-58.
65. Parra, E. R., et al., Validation of multiplex immunofluorescence panels using multispectral microscopy for immune-profiling of formalin-fixed and paraffin-embedded human tumor tissues. Scientific Reports, 2017. 7 (1): p. 13380.
66. Blom, S., et al., Systems pathology by multiplexed immunohistochemistry and whole-slide digital image analysis. Scientific reports, 2017. 7 (1): p. 15580-15580.
67. Mansfield, J., et al., Validation of multiplex immunofluorescence for use in analysis of tumour infiltrating lymphocytes. Vol. 3. 2015. p 411.
68. Gorris, M. A. J., et al., Eight-Color Multiplex Immunohistochemistry for Simultaneous Detection of Multiple Immune Checkpoint Molecules within the Tumor Microenvironment. J Immunol, 2018. 200 (1): p. 347-354.
69. Lesinski, G. B., et al., Interleukin-6/STAT3 Signaling is Prominent and Associated with Reduced Overall Survival in p 16 Negative Oropharyngeal Squamous Cell Carcinoma. Head Neck Pathol, 2018.
70. Harper, S. J. F., et al., CD8 T-cell recognition of acquired alloantigen promotes acute allograft rejection. Proceedings of the National Academy of Sciences of the United States of America, 2015. 112 (41): p. 12788-12793.
71. Rocha, P. N., et al., Effector mechanisms in transplant rejection. Immunol Rev, 2003. 196: p. 51-64.
72. Le Moine, A., M. Goldman, and D. Abramowicz, Multiple pathways to allograft rejection. Transplantation, 2002. 73 (9): p. 1373-81.
73. Bestard, O., et al., Presence of FoxP3+ regulatory T Cells predicts outcome of subclinical rejection of renal allografts. J Am Soc Nephrol, 2008. 19 (10): p. 2020-6.
74. Raimondi, G., et al., Mammalian target of rapamycin inhibition and alloantigen-specific regulatory T cells synergize to promote long-term graft survival in immunocompetent recipients. J Immunol, 2010. 184 (2): p. 624-36.
75. Liao, T., et al., In Vivo Attenuation of Antibody-Mediated Acute Renal Allograft Rejection by Ex Vivo TGF-β-Induced CD4(+)Foxp3(+) Regulatory T Cells. Frontiers in immunology, 2017. 8: p. 1334-1334.
76. Chung, B. H., et al., Clinical significance of the ratio between FOXP3 positive regulatory T cell and interleukin-17 secreting cell in renal allograft biopsies with acute T-cell-mediated rejection. Immunology, 2012. 136 (3): p. 344-51.
77. Hua, J., et al., Pathological conversion of regulatory T cells is associated with loss of allotolerance. Sci Rep, 2018. 8 (1): p. 7059.
78. Sehrawat, S. and B. T. Rouse, Interplay of Regulatory T Cell and Th17 Cells during Infectious Diseases in Humans and Animals. Frontiers in Immunology, 2017. 8: p. 341.
79. Komatsu, N., et al., Pathogenic conversion of Foxp3+ T cells into TH1 7 cells in autoimmune arthritis. Nat Med, 2014. 20 (1): p. 62-8.
80. Loverre, A., et al., IL-17 expression by tubular epithelial cells in renal transplant recipients with acute antibody-mediated rejection. Am J Transplant, 2011. 11 (6): p. 1248-59.
81. Spain, L., et al., Acute renal allograft rejection after immune checkpoint inhibitor therapy for metastatic melanoma. Ann Oncol, 2016. 27 (6): p. 1135-7.
82. Alhamad, T., et al., Checkpoint Inhibitors in Kidney Transplant Recipients and the Potential Risk of Rejection. Am J Transplant, 2016. 16 (4): p. 1332-3.
83. Goldman, J. W., et al., PD1 checkpoint inhibition in solid organ transplants: 2 sides of a coin—case report. BMC Nephrology, 2018. 19 (1): p. 210.
84. Lipson, E. J., et al., Tumor Regression and Allograft Rejection after Administration of Anti-PD-1. N Engl J Med, 2016. 374 (9): p. 896-8.
85. Pilat, N., M. H. Sayegh, and T. Wekerle, Costimulatory pathways in transplantation. Seminars in Immunology, 2011. 23 (4): p. 293-303.
86. Tanaka, K., et al., PDL1 is required for peripheral transplantation tolerance and protection from chronic allograft rejection. Journal of immunology (Baltimore, Md.: 1950), 2007. 179 (8): p. 5204-5210.
87. Schutte-Nutgen, K., et al., Divergent Function of Programmed Death-Ligand 1 in Donor Tissue versus Recipient Immune System in a Murine Model of Bronchiolitis Obliterans. The American Journal of Pathology, 2017. 187 (6): p. 1368-1379.
88. Li, Y. M., et al., Soluble Tim-3 and Gal-9 are associated with renal allograft dysfunction in kidney transplant recipients: A cross-sectional study. Int Immunopharmacol, 2018. 55: p. 330-335.
89. Lv, K., et al., Galectin-9 administration ameliorates CVB3 induced myocarditis by promoting the proliferation of regulatory T cells and alternatively activated Th2 cells. Clin Immunol, 2011. 140 (1): p. 92-101.
90. Naka, E. L., et al., Detection of the Tim-3 ligand, galectin-9, inside the allograft during a rejection episode. Int Immunopharmacol, 2009. 9 (6): p. 658-62.
91. Qiao, X., et al., Increased expression of Tim-3 and its ligand Galectin-9 in rat allografts during acute rejection episodes. Biochem Biophys Res Commun, 2014. 445 (3): p. 542-8.
92. Zhang, A.-B., et al., Exosome-derived galectin-9 may be a novel predictor of rejection and prognosis after liver transplantation. Journal of Zhejiang University. Science. B, 2019. 20 (7): p. 605-612.
93. Zhang, Y., et al., Galectin-9 induced myeloid suppressor cells expand regulatory T cells in an IL-10-dependent manner in CVB3-induced acute myocarditis. International journal of molecular sciences, 2014. 15 (3): p. 3356-3372.
94. Bruneval, P., et al., The XIIIth Banff Conference on Allograft Pathology: The Banff 2015 Heart Meeting Report: Improving Antibody-Mediated Rejection Diagnostics: Strengths, Unmet Needs, and Future Directions. Am J Transplant, 2017. 17 (1): p. 42-53.
95. Salehi, S. and E. F. Reed, The divergent roles of macrophages in solid organ transplantation. Current opinion in organ transplantation, 2015. 20 (4): p. 446-453.
96. Liu, Y., M. Kloc, and X. C. Li, Macrophages as Effectors ofAcute and Chronic Allograft Injury. Current transplantation reports, 2016. 3 (4): p. 303-312.
97. Mannon, R. B., Macrophages: contributors to allograft dysfunction, repair, or innocent bystanders? Current opinion in organ transplantation, 2012. 17 (1): p. 20-25.
98. van den Bosch, T. P. P., et al., Targeting the Monocyte-Macrophage Lineage in Solid Organ Transplantation. Frontiers in immunology, 2017. 8: p. 153-153.
99. Colvin, M. M., et al., Antibody-mediated rejection in cardiac transplantation: emerging knowledge in diagnosis and management: a scientific statement from the American Heart Association. Circulation, 2015. 131 (18): p. 1608-39.
100. Edwards, L. A., et al., Chronic Rejection of Cardiac Allografts Is Associated With Increased Lymphatic Flow and Cellular Trafficking. Circulation, 2018. 137 (5): p. 488-503.
101. Tsuchimoto, A., et al., The potential role of perivascular lymphatic vessels in preservation of kidney allograft function. Clinical and Experimental Nephrology, 2017. 21 (4): p. 721-731.
102. Vass, D. G., J. Hughes, and L. P. Marson, Restorative and Rejection-Associated Lymphangiogenesis After Renal Transplantation: Friend or Foe? Transplantation, 2009. 88 (11).
103. Igarashi, T., et al., Scoring of PD-L1 expression intensity on pulmonary adenocarcinomas and the correlations with clinicopathological factors. ESMO Open, 2016. 1 (4): p. e000083.
104. Janowczyk, A., et al., High-Throughput Biomarker Segmentation on Ovarian Cancer Tissue Microarrays via Hierarchical Normalized Cuts. IEEE Transactions on Biomedical Engineering, 2012. 59 (5): p. 1240-1252.
105. Maragos, P., Pattern spectrum and multiscale shape representation. IEEE Trans Pattern Anal Mach Intell. Pattern Analysis and Machine Intelligence, IEEE Transactions on, 1989. 11: p. 701-716.
106. Vincent, L., “Granulometries and Opening Trees”. Fundamenta Informaticae, 2000. 41 (1-2): p. 57-90.
107. Haralick, R. M., Statistical and structural approaches to texture. Proceedings of the IEEE, 1979. 67 (5): p. 786-804.
108. Haralick, R. M., K. Shanmugam, and I. Dinstein, Textural Features for Image Classification. IEEE Transactions on Systems, Man, and Cybernetics, 1973. SMC-3 (6): p. 610-621.
109. Zhao, J., et al., Learning from heterogeneous temporal data in electronic health records. Journal of Biomedical Informatics, 2017. 65: p. 105-119.
110. Ordonez, P., et al. Using Modified Multivariate Bag-of-Words Models to Classify Physiological Data. in 2011 IEEE 11th International Conference on Data Mining Workshops. 2011.
111. Orphanou, K., A. Stassopoulou, and E. Keravnou, Temporal abstraction and temporal Bayesian networks in clinical domains: A survey. Artificial Intelligence in Medicine, 2014. 60 (3): p. 133-149.
112. Liu, C., et al., Temporal Phenotyping from Longitudinal Electronic Health Records: A Graph Based Framework, in Proceedings of the 21th ACM SIGKDD International Conference on Knowledge Discovery and Data Mining. 2015, Association for Computing Machinery: Sydney, NSW, Australia. p. 705-714.
113. Luo, G., A Roadmap for Semi-automatically Extracting Predictive and Clinically Meaningful Temporal Features from Medical Data for Predictive Modeling. Global Transitions, 2019. 1.
114. Rajkomar, A., et al., Scalable and accurate deep learning with electronic health records. npj Digital Medicine, 2018. 1 (1): p. 18.
115. Wu, S., et al., Modeling asynchronous event sequences with RNNs. Journal of Biomedical Informatics, 2018. 83: p. 167-177.
116. Bernardini, M., et al., Discovering the Type 2 Diabetes in Electronic Health Records Using the Sparse Balanced Support Vector Machine. IEEE J Biomed Health Inform, 2020. 24 (1): p. 235-246.
117. Cortes, C. and V. Vapnik, Support-vector networks. Machine Learning, 1995. 20 (3): p. 273-297.
118. Li, X., C. X. Ling, and H. Wang, The Convergence Behavior of Naive Bayes on Large Sparse Datasets. ACM Trans. Knowl. Discov. Data, 2016. 11 (1): p. Article 10.
119. Zheng, Z., et al. Sparse Weighted Naive Bayes Classifier for Efficient Classification of Categorical Data. in 2018 IEEE Third International Conference on Data Science in Cyberspace (DSC). 2018.
120. Pham, T., et al., Predicting healthcare trajectories from medical records: A deep learning approach. Journal of Biomedical Informatics, 2017. 69: p. 218-229.
121. Ma, F., et al., Dipole: Diagnosis Prediction in Healthcare via Attention-based Bidirectional Recurrent Neural Networks, in Proceedings of the 23rd ACM SIGKDD International Conference on Knowledge Discovery and Data Mining. 2017, Association for Computing Machinery: Halifax, NS, Canada. p. 1903-1911.
122. Luo, G., et al., Using Temporal Features to Provide Data-Driven Clinical Early Warnings for Chronic Obstructive Pulmonary Disease and Asthma Care Management: Protocol for a Secondary Analysis. JMIR research protocols, 2019. 8 (6): p. e13783-e13783.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for detecting an increased propensity for heart transplant rejection, graft dysfunction, or organ failure, the method comprising:
(a) providing a sample from a subject who has received a transplant from a donor;
(b) obtaining an immunophenotype to establish an immune cell infiltration and immune effector profile for detecting risk of serious allograft injury and
(c) diagnosing, predicting, or monitoring a transplant status or outcome of the subject who has received the transplant by determining the proportional expression levels of immune cell markers present in said transplant, said markers being at least five of CD3, CD8, FoxP3, CD68, PDL1, IL17, Gal-9, CD4, CD20, CD86, D2-40, and CD31 and indicating increase risk of transplant rejection, graft dysfunction or organ failure.

2. The method according to claim 1, wherein immunophenotype is examined using immunocytochemistry, immunoblotting, flow cytometry, or fluorescence-activated cell sorting.

3. The method of claim 1, wherein the biological sample is endomyocardial biopsy (EMB).

4. The method of claim 1, wherein said rejection is clinically silent rejection.

5. The method of claim 1, further comprising administering one or more immunosuppressive drug.

6. The method of claim, 1, wherein the diagnosing, predicting, or monitoring transplant status or outcome comprises treating a transplant rejection in a subject in need thereof.

7. The method of claim 1, wherein the diagnosing, predicting, or monitoring transplant status or outcome comprises determining, modifying, or maintaining an immunosuppressive regimen based on modulation therapeutic targets differentially expressed in said EMB.

8. A method for identifying cardiac transplant tissue rejection in a human subject, said method comprising: determining a first immunophenotype profile in an EMB sample taken from said human subject, wherein said first immunophenotype profile comprises the protein expression levels of CD3, CD8, FoxP3, IL17, PDL1, Gal-9, CD4, CD20, CD68, CD86, D2-40, and CD31; and comparing said first immunophenotype profile to a second immunophenotype profile, wherein said second immunophenotype profile comprises the protein expression levels of CD3, CD8, FoxP3, IL17, PDL1, Gal-9, CD4, CD20, CD68, CD86, D2-40, and CD31 obtained from EMB samples collected from a human cardiac transplant population that does not have cardiac transplant tissue rejection, wherein a statistically significant alteration in proportional expression and intensity distribution patterns distributions of CD3, CD8, FoxP3, IL17, PDL1, Gal-9, CD4, CD20, CD68, CD86, D2-40, and CD31 in said first immunophenotype profile compared to said second immunophenotype profile is indicative of cardiac transplant tissue rejection in said human subject.

9. The method according to claim 8, wherein immunophenotype is examined using immunocytochemistry, immunoblotting, flow cytometry, or fluorescence-activated cell sorting.

10. The method of claim 8, wherein said rejection is clinically silent rejection.

11. The method of claim 8, further comprising administering one or more immunosuppressive drug.

12. A method for identifying a subject at risk for future cardiac transplant tissue rejection, said method comprising: determining a first immunophenotype profile in an EMB sample taken from said human subject, wherein said first immunophenotype profile comprises the protein expression levels of CD3, CD8, FoxP3, IL17, PDL1, Gal-9, CD4, CD20, CD68, CD86, D2-40, and CD31; and comparing said first immunophenotype profile to a second immunophenotype profile, wherein said second immunophenotype profile comprises the protein expression levels of CD3, CD8, FoxP3, IL17, PDL1, Gal-9, CD4, CD20, CD68, CD86, D2-40, and CD31 obtained from EMB samples collected from a human cardiac transplant population that does not have cardiac transplant tissue rejection, wherein a statistically significant alteration in proportional expression and intensity distribution patterns distributions of CD3, CD8, FoxP3, IL17, PDL1, Gal-9, CD4, CD20, CD68, CD86, D2-40, and CD31 in said first immunophenotype profile compared to said second immunophenotype profile is indicative of increased risk of cardiac transplant tissue rejection in said human subject.

13. The method according to claim 12, wherein immunophenotype is examined using immunocytochemistry, immunoblotting, flow cytometry, or fluorescence-activated cell sorting.

14. The method of claim 12, wherein said rejection is clinically silent rejection, and said method further comprises administering one or more immunosuppressive drug.

15. (canceled)

16. A computer-implemented method for detection of an increased risk of cardiac transplant rejection in a subject in need thereof, comprising executing on a processor the steps of: performing quantitative pattern analysis of immunofluorescence data corresponding to an immunophenotype in an EMB indicative of undesirable immune cell infiltration correlated with allograft rejection to determine a level of spatial heterogeneity or similarity with an immunophenotype in standard subject not experiencing allograft rejection; and assigning an allograft rejection risk based on the level of spatial heterogeneity or similarity determined during said performing step.

17. The method of claim 16, wherein that immunophenotype is assigned based on differential proportional expression levels of immune cell markers in said EMB, indicative of future transplant rejection risk.

18. The method of claim 16, wherein said immune cell markers comprise at least three of CD3, CD8, FoxP3, IL17, PD-L1, Gal-9, CD4, CD20, CD68, CD86, D2-40, and CD31.

19. The method of claim 16, wherein said markers are at least CD3, CD8, CD68, PD-L1, and FoxP3.

20. The method of claim 16, wherein expression levels of each of CD3, CD8, FoxP3, IL17, PD-L1, Gal-9, CD4, CD20, CD68, CD86, D2-40, and CD31 are determined.

21. The method of claim 16, wherein cellular spatial and patterns are mapped, thereby characterizing in-situ interactions of immune cells and effectors.

22. The method of claim 21, further comprising spatial analysis of lymphocyte location based on compartment segmentation, and assessment of interactions with myocytes via proximity graphs.
